SEQUENCE LISTING (32 sequences)

SEQ ID NO: 1 - protein, 219 aa, Mus musculus
DVVMTQTPLTLSVTIGQPASISCKSSQSLLDTDGKTYLNWLLQRPGQSPKRLIYLVSKLDSGVPDRFTGSGSGTDFTLKISRVEAEDLGVYYCWQGTHFPQTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 443 aa, Mus musculus
EVQLQQSGPELVKPGASVKISCKASGYSFTGYFMHWVKQRHVQSLEWIGRINPYNGATNYNQIFKDKASLTLDKSSSTAYMELHSLTSEDSAVYYCASGGHYYGNVDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 3 - protein, 138 aa, Mus musculus
MGWSWIFLFLLSGTAGVLSEVQLQQSGPELVKPGASVKISCKASGYSFTGYFMHWVKQRHVQSLEWIGRINPYNGATNYNQIFKDKASLTLDKSSSTAYMELHSLTSEDSAVYYCASGGHYYGNVDYWGQGTTLTVSS

SEQ ID NO: 4 - protein, 131 aa, Mus musculus
MSPAQFLLLLVLWIRETNGDVVMTQTPLTLSVTIGQPASISCKSSQSLLDTDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 5 - protein, 16 aa, Mus musculus
KSSQSLLDTDGKTYLN

SEQ ID NO: 6 - protein, 7 aa, Mus musculus
LVSKLDS

SEQ ID NO: 7 - protein, 9 aa, Mus musculus
WQGTHFPQT

SEQ ID NO: 8 - protein, 5 aa, Mus musculus
GYFMH

SEQ ID NO: 9 - protein, 17 aa, Mus musculus
RINPYNGATNYNQIFKD

SEQ ID NO: 10 - protein, 10 aa, Mus musculus
GGHYYGNVDY

SEQ ID NO: 11 - protein, 7 aa, Mus musculus
GYSFTGY

SEQ ID NO: 12 - protein, 6 aa, Mus musculus
NPYNGA

SEQ ID NO: 13 - DNA, 393 bp, Mus musculus
  source 1..393: mol_type = genomic DNA
atgagtcctgcccagttcctgcttctgttagtgctctggattcgggaaaccaacggtgatgttgtgatgacccagactccactcactttgtcggttaccattggacaaccagcctccatctcttgcaagtcaagtcagagcctcttagacactgatggaaagacatatttgaattggttattacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 14 - DNA, 414 bp, Mus musculus
  source 1..414: mol_type = genomic DNA
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctacttcatgcactgggtgaagcaaaggcatgtacagagccttgagtggattggacgtattaatccttacaatggtgctactaactacaaccagattttcaaagacaaggccagcttgactttagataagtcctccagcacagcctacatggagctccacagcctgacatctgaggactctgcagtctattactgtgcaagcggagggcattactacggcaacgttgactactggggccaaggcaccactctcacagtctcctca

SEQ ID NO: 15 - protein, 16 aa, unidentified
KSSQSLLDTDGKTYLN

SEQ ID NO: 16 - protein, 7 aa, unidentified
LVSKLDS

SEQ ID NO: 17 - protein, 9 aa, unidentified
WQGTHFPQT

SEQ ID NO: 18 - protein, 5 aa, unidentified
GYFMH

SEQ ID NO: 19 - protein, 17 aa, unidentified
RINPYNGATNYNQIFKD

SEQ ID NO: 20 - protein, 10 aa, unidentified
GGHYYGNVDY

SEQ ID NO: 21 - protein, 7 aa, unidentified
GYSFTGY

SEQ ID NO: 22 - protein, 6 aa, unidentified
NPYNGA

SEQ ID NO: 23 - protein, 246 aa, unidentified
QVQLVQSGPEVKKPGASVKVSCKASGYTFTGYFMHWVKQTPGQGLEWIGRINPYNGATNYNQIFKDRVTMTLDKSSSTAYMELHSLTSDDTAVYYCASGGHYYGNVDYWGQGTLLTVSSGGGGSGGGGSGGGGSDVVMTQSPLSLSVTLGQPASISCKSSQSLLDTDGKTYLNWLQQRPGQSPRRLIYLVSKLDSGVPDRFSGSGSGTDFTLKISRVEAEDVGVYYCWQGTHFPQTFGGGTKLEIK

SEQ ID NO: 24 - protein, 246 aa, unidentified
QVQLVQSGPEVKKPGASVKISCKASGYTFTGYFMHWVKQTHGQGLEWIGRINPYNGATNYNQIFKDRVTLTLDTSSSTAYMELPRLRSDDTAVYYCASGGHYYGNVDYWGQGTLVTVSSGGGGSGGGGSGGGGSDVVMTQSPLSLPVTLGQPASISCKSSQSLLDTDGKTYLNWLLQRPGQSPRRLIYLVSKLDSGVPDRFSGSGSGTDFTLKISRVEAEDVGVYYCWQGTHFPQTFGGGTKLEIK

SEQ ID NO: 25 - protein, 246 aa, unidentified
QVQLVQSGAEVIKPGASVKISCKASGYTFTGYFMHWVKQTPGQGLEWMGRINPYNGATNYNQIFKDRVTLTLDTSSSTAYMELSRLRSDDTAVYYCASGGHYYGNVDYWGQGTLLTVSSGGGGSGGGGSGGGGSDVVMTQSPLSLPVTLGQPASISCKSSQSLLDTDGKTYLNWLQQRPGQSPRRLIYLVSKLDSGVPDRFSGSGSGTDFTLKISRVEAEDVGVYYCWQGTHFPQTFGGGTKLEIK

SEQ ID NO: 26 - protein, 246 aa, unidentified
QVQLVQSGPEVIKPGASVKVSCKASGYTFTGYFMHWVRQTPGQGLEWIGRINPYNGATNYNQIFKDRVTMTLDTSSSTAYMELSSLRSDDTAVYYCASGGHYYGNVDYWGQGTLVTVSSGGGGSGGGGSGGGGSDVVMTQSPLSLSVTLGQPASISCKSSQSLLDTDGKTYLNWLQQRPGQSPRRLIYLVSKLDSGVPDRFSGSGSGTDFTLKISRVEAEDVGVYYCWQGTHFPQTFGGGTKLEIK

SEQ ID NO: 27 - protein, 246 aa, unidentified
QVQLVQSGPEVKKPGASVKVSCKASGYTFTGYFMHWVKQTPGQGLEWIGRINPYNGATNYNQIFKDRVTLTLDTSISTAYMELPRLRSDDTAVYYCASGGHYYGNVDYWGQGTLLTVSSGGGGSGGGGSGGGGSDVVMTQSPLSLSVTLGQPASISCKSSQSLLDTDGKTYLNWLLQRPGQSPRRLIYLVSKLDSGVPDRFSGSGSGTDFTLKISRVEAEDVGVYYCWQGTHFPQTFGGGTKLEIK

SEQ ID NO: 28 - protein, 246 aa, unidentified
QVQLVQSGPEVKKPGASVKVSCKASGYSFTGYFMHWVRQTHGQGLEWIGRINPYNGATNYNQIFKDRVTLTLDKSISTAYMELSRLTSDDTAVYYCASGGHYYGNVDYWGQGTLVTVSSGGGGSGGGGSGGGGSDVVMTQSPLSLSVTLGQPASISCKSSQSLLDTDGKTYLNWLQQRPGQSPRRLIYLVSKLDSGVPDRFSGSGSGTDFTLKISRVEAEDVGVYYCWQGTHFPQTFGGGTKLEIK

SEQ ID NO: 29 - protein, 246 aa, unidentified
QVQLVQSGPEVIKPGASVKISCKASGYTFTGYFMHWVRQAPGQGLEWIGRINPYNGATNYNQIFKDRVTLTLDTSSSTAYMELSRLRSDDTAVYYCASGGHYYGNVDYWGQGTLLTVSSGGGGSGGGGSGGGGSDVVMTQSPLSLSVTLGQPASISCKSSQSLLDTDGKTYLNWLLQRPGQSPRRLIYLVSKLDSGVPDRFSGSGSGTDFTLKISRVEAEDVGVYYCWQGTHFPQTFGGGTKLEIK

SEQ ID NO: 30 - protein, 246 aa, unidentified
QVQLVQSGPEVIKPGASVKISCKASGYTFTGYFMHWVKQTHGQGLEWMGRINPYNGATNYNQIFKDRVTMTLDKSSSTAYMELSSLRSDDTAVYYCASGGHYYGNVDYWGQGTLVTVSSGGGGSGGGGSGGGGSDVVMTQSPLSLPVTLGQPASISCKSSQSLLDTDGKTYLNWLLQRPGQSPRRLIYLVSKLDSGVPDRFSGSGSGTDFTLKISRVEAEDVGVYYCWQGTHFPQTFGGGTKLEIK

SEQ ID NO: 31 - protein, 246 aa, unidentified
QVQLVQSGPEVIKPGASVKVSCKASGYTFTGYFMHWVRQTPGQGLEWIGRINPYNGATNYNQIFKDRVTLTLDKSSSTAYMELSSLTSDDTAVYYCASGGHYYGNVDYWGQGTLVTVSSGGGGSGGGGSGGGGSDVVMTQSPLSLSVTLGQPASISCKSSQSLLDTDGKTYLNWLLQRPGQSPRRLIYLVSKLDSGVPDRFSGSGSGTDFTLKISRVEAEDVGVYYCWQGTHFPQTFGGGTKLEIK

SEQ ID NO: 32 - protein, 246 aa, unidentified
QVQLVQSGPEVKKPGASVKVSCKASGYTFTGYFMHWVKQTPGQGLEWIGRINPYNGATNYNQIFKDRVTMTLDTSSSTAYMELSRLRSDDTAVYYCASGGHYYGNVDYWGQGTLVTVSSGGGGSGGGGSGGGGSDVVMTQSPLSLPVTLGQPASISCKSSQSLLDTDGKTYLNWLQQRPGQSPRRLIYLVSKLDSGVPDRFSGSGSGTDFTLKISRVEAEDVGVYYCWQGTHFPQTFGGGTKLEIK

(19) United States
(12) Patent Application Publication
(10) Pub. No.: US 2026/0274969 A1
(43) Pub. Date: Sep. 17, 2026

(54) GFRA2 AS A CANDIDATE IMMUNOTHERAPEUTIC TARGET IN NEUROBLASTOMA AND EWING SARCOMA

(71) Applicants: Texas Tech Univesity, Lubbock, TX (US); The Children's Hospital of Philadelphia, Philadellphia, PA (US); University of Pittsburgh-of the Commonwealth System of Higher Education, Pittsburgh, PA (US)

(72) Inventors: Michelle E. Keyel, Lubbock, TX (US); Charles Patrick Reynolds, Lubbock, TX (US); John M. Maris, Philadelphia, PA (US); Amber K. Hamilton, Philadelphia, PA (US); Sharon J. Diskin, Philadelphia, PA (US); Wei Li, Pittsburgh, PA (US); Seungmin Shin, Pittsburgh, PA (US); Maggie Hines, Pittsburgh, PA (US)

(73) Assignees: Texas Tech University System, Lubbock, TX (US); The Children's Hospital of Philadelphia, Philadephia, PA (US); University of Pittsburgh-of the Commonwealth System of Higher Education, Pittsburgh, PA (US)

(21) Appl. No.: 19/687,864

(22) Filed: May 26, 2026

Related U.S. Application Data
(63) Continuation-in-part of application No. 18/694,966, filed on Jan. 18, 2024.
(60) Provisional application No. 63/439,799, filed on Jan. 18, 2023.
(60) Provisional application No. 63/811,175, filed on May 23, 2025.

(51) Int. Cl. C07K 16/28; A61K 47/68; A61P 35/00

(52) U.S. Cl. CPC C07K 16/2863; A61K 47/6803; A61K 47/68031; A61K 47/68035; A61K 47/68037; A61K 47/6849; A61P 35/00; C07K 2317/24; C07K 2317/31

(57) ABSTRACT

The invention relates to a humanized antibody that binds to GDNF family receptor alpha 2 (GFRA2). The antibody may be in a drug conjugated form. The invention also relates to methods of treating or preventing a cancer in a subject by administering to the subject an antibody described herein. Additionally, the invention relates to chimeric antigen receptor T-cells that express an antibody described herein and methods of treating or preventing a cancer in a subject by administering them to the subject.

### CROSS-REFERENCE TO RELATED APPLICATIONS

The application is a continuation-in-part application of U.S. patent application Ser. No. 18/694,966, filed on Mar. 23, 2024, which is a U.S. national stage application of International Patent Application No. PCT/US2024/011995, filed on Jan. 18, 2024, which claims priority to U.S. Provisional Patent Application No. 63/439,799, filed on Jan. 18, 2023. This application also claims priority to U.S. Provisional Patent Application No. 63/811,175, filed on May 23, 2025. The entirety of each of the aforementioned applications is incorporated herein by reference.

### SEQUENCE DISCLOSURE STATEMENT

Pursuant to 37 C.F.R. § 1.834, Applicants have submitted a sequence listing in XML format (“Sequence Listing”). The name of the file containing the Sequence Listing is “AF13368.P049P1.xml”. The date of the creation of the Sequence Listing is May 26, 2026. The size of the Sequence Listing is 32,000 bytes. Applicants hereby incorporate by reference the material in the Sequence Listing.

### BACKGROUND

Current methods of treating various cancers have numerous limitations, such as limited therapeutic options and cancer cell resistance. Numerous embodiments of the present disclosure aim to address the aforementioned limitations.

### SUMMARY

In some embodiments, the present disclosure pertains to a humanized antibody that binds to GDNF family receptor alpha 2 (GFRA2). In some embodiments, the antibody is in a modified form. In some embodiments, the modified form includes, without limitation, a chimeric form, a humanized form, a drug conjugated form, a bispecific form, a labeled form, a fragmented form, or combinations thereof.

Additional embodiments of the present disclosure pertain to methods of treating or preventing a cancer in a subject by administering to the subject an antibody of the present disclosure. Further embodiments of the present disclosure pertain to chimeric antigen receptor T-cells (CAR T-cells) that express an antibody of the present disclosure. Additional embodiments of the present disclosure pertain to methods of treating or preventing a cancer in a subject by administering to the subject a CAR T-cell of the present disclosure.

### DESCRIPTION OF THE DRAWINGS

FIGS. 1A-1B provide GD2 expression profiles by flow cytometry on neuroblastoma (NB) patient-derived cell lines. Using labeled dinutuximab, Applicant assessed % GD2-positive (FIG. 1A) and median antigen surface expression (median fluorescence intensity=MFI, FIG. 1B) of NB cell lines obtained at diagnosis and at time of progressive disease, some of which were obtained at time of demise from progressive disease. These data suggest there are 3 distinct groups of NB with significantly different % positive for GD2 (<50%, 50% to 80%, and >80%). Density of antibody binding is significantly higher for the high % positive lines compared to those with lower % positive cells. This provides a potential framework on which to build and determine parameters (% positive, binding intensity, or both) that determine response and resistance to dinutuximab.

FIGS. 1C-1D show binding patterns for an antibody that was highly specific for neuroblastoma (HSAN) compared to dinutuximab (GD2) across a panel of 24 neuroblastoma patient-derived cell lines (PDCLs) from the Children's Oncology Group (COG) repository established from 24 different patients. Dinutuximab and HSAN were directly fluorescently labeled as per FIG. 2. Data was obtained by analyzing labeled cells on a flow cytometer. FIG. 1C shows % Cells Antibody-positive. FIG. 1D shows median antigen surface expression per cell line (as median fluorescence intensity=MFI). Most GD2-low and GD2 negative cell lines show strong HSAN binding. Dark blue bars=dinutuximab, light blue bars=HSAN.

FIG. 2 illustrates a multi-color flow cytometry assay to quantify GD2 on NB cells from marrow and blood. Labeled anti-CD45 monoclonal antibodies (mabs) were used to gate out hematopoietic cells. A fluorescently labeled neuroblastoma (NB)-specific, non-GD2-binding monoclonal antibody (HSAN) was used to gate on NB cells. Applicant then quantified % positive and density of dinutuximab binding (median fluorescence intensity=MFI) using labeled dinutuximab. The right panel shows example results from patient bone marrows with high- or low-positive dinutuximab-binding using Applicant's methodology. Histograms are of dinutuximab staining for all HSAN+ cells (i.e. NB) in the marrow.

FIGS. 3A-3D illustrate the quantification of dinutuximab binding in clinical samples by flow cytometry. Using the assay in FIG. 2, Applicant assessed dinutuximab binding to neuroblastoma (NB) cells in patient samples. FIG. 3A provides cytograms showing that only NB cells (gating out CD45+ cells, gating on HSAN+). Patient at diagnosis (DX) had high dinutuximab binding while patients with progressive disease (PD) showed low dinutuximab binding. The quadrants are: a. NB cells negative for GD2; b. NB cells positive for GD2; and c+d. non-NB cells after gating. FIG. 3B shows samples from patients (8 tumors, 64 bone marrow (BM) samples, and 12 blood samples) that were analyzed for % GD2-positive and Median Fluorescence Intensity (MFI). These data demonstrate that patients at diagnosis (DX) and progressive disease (PD) have tumor cells that were weak or negative for GD2 (all NB cells were identified by HSAN). FIG. 3C shows a graph comparing % GD2 positive vs GD2 MFI for NB cells (gated on by HSAN) in bone marrow. FIG. 3D summarizes % of samples in each of four groups defined by low to high dinutuximab binding. Note the higher % of PD samples with low GD2 compared to samples obtained from patients pre-therapy (DX).

FIGS. 4A-4C show that HSAN is a GFRA2 antibody and GRFA2 has limited normal tissue expression. FIG. 4A shows quantitative polymerase chain reaction (pPCR) analysis of a 72 hr siRNA knockdown of GFRA2 in the NB cell line COG-N-534. FIG. 4B shows GFRA2 polyclonal antibody and HSAN surface staining using flow cytometry in COG-N-534 after 96 hr siRNA GFRA2 knockdown. FIG. 4C summarizes data that neuroblastomas express higher amounts of GFRA2 mRNA compared to healthy tissues (attained from Yarmarkovich et al., Nature 623, 820-827, (2023). The data represent analysis of NCI TARGET data of 153 NB tumors compared to 1643 normal tissues in GTEx.

FIGS. 5A-5B show additional data demonstrating that HSAN binds to GFRA2. Applicant conducted an additional experiment to demonstrate that HSAN binds to GFRA2. HEK293 cells (GFRA2-negative cells) were plated the day before transfection. Cells were then transfected with either GFRA2 plasmid DNA or empty vector control plasmid DNA using the MegaTran 2.0 plasmid transfection reagent. Medium was changed after 24 hours of transfection. GFRA2 surface expression was assessed by flow cytometry 72 hours post-transfection on a BD Fortessa flow cytometer. Untransfected parental HEK cells, empty vector transfected, or GFRA2 transfected cells were stained with the HSAN antibody directly conjugated to Alexa Fluor 488, a commercially available anti-GFRA2 antibody with an anti-goat CF 594 secondary antibody, or the secondary antibody alone. Flow data were analyzed using FlowJo software. This experiment demonstrated that cells that do not express GFRA2 were induced to express GFRA2 on the cell surface by transfection of GFRA2 DNA, detectable by an antibody to GFRA2 and also by the HSAN monoclonal antibody.

FIGS. 6A-6B provide data related to the cell surface expression of GFRA2 in Neuroblastoma. FIG. 6A provides immunofluorescence showing colocalization of GFRA2 with cell surface protein Cadherin. FIG. 6B provides flow cytometry showing cell surface expression in subsets of cell lines and xenografts.

FIGS. 7A-7C demonstrate that GFRA2 is expressed in Ewing Sarcoma (EWS) and is a EWSR1::FLI1 target gene. FIG. 7A provides data related to cell surface validation of GFRA2 in EWS cell lines (N=4) by flow cytometry (CHLA9, CHLA10 not shown). FIG. 7B provides data demonstrating that siRNA knockdown of EWSR1::FLI1 results in down-regulation of GFRA2. The results suggest that GFRA2 is a direct EWSR1::FLI1 transcriptional target. FIG. 7C provide validation of GFRA2 knockdown in response to siEWSR1::FLI1 at the protein level by western blotting.

FIGS. 8A-8C illustrate the humanization of an HSAN antibody targeting GFRA2. FIG. 8A shows that complementary determining region (CDR) graft humanization from mouse to human compromised binding. FIG. 8B illustrates that back mutation from mouse to human identified regions critical for binding. FIG. 8C shows that combination of mouse and human sequence yielded a synthetic humanized HSAN that is ~94% human.

FIGS. 9A-9D illustrate the reformatting and characterization of a humanized HSAN anti-GFRA2 antibody scFv fragment (SynHuHSAN scFv) into a human IgG1 (SynHuHSAN human IgG1).

FIGS. 10A-10B illustrate the internalization of GFRA2 by SynHuHSAN human IgG1. The HSAN antibody was conjugated to pH sensitive Fab-flour. FIG. 10A shows that IMR5 (GFRA2+ NB) internalizes the HSAN antibody, as shown by fluorescence over time that is absent in the IgG control. FIG. 10B shows that SK-N-Be (2) C (GFRA2-NB) does not internalize the HSAN antibody, as reflected in no fluorescence in either HSAN or IgG control.

FIG. 11 shows cell binding specificity for SynhuHSAN IgG1, as measured by flow cytometry.

FIGS. 12A-12C illustrate the generation and validation of SynHuHSAN IgG-antibody drug conjugates (ADCs) by an inter-disulfide bond conjugation method.

FIGS. 13A-13D provide data indicating that SynHuHSAN IgG-ADC shows potent and specific cytotoxicity. SynHuHSAN IgG was conjugated to pyrrolobenzodiazepine (PBD) to form a GFRA2 directed antibody drug conjugate (GFRA2-ADC). FIGS. 13A-13B demonstrate that GFRA2-ADC shows potent cytotoxicity in GFRA2⁺ neuroblastoma cell lines compared to PBD control. FIG. 13C demonstrate that GFRA2-ADC shows potent cytotoxicity in GFRA2⁺ EWS cell lines compared to PBD control. FIG. 13D demonstrate that GFRA2-ADC shows no cytotoxicity in GFRA2 melanoma cell lines with PBD control, showing potent cytotoxicity.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current methods of treating various cancers have numerous limitations, such as limited therapeutic options and cancer cell resistance. For instance, an existing method of treating neuroblastoma (NB) involves the administration of dinutuximab, an anti-GD2 antibody to patients. However, treatment with dinutuximab causes loss of binding as a mechanism of cancer cell resistance. Additionally, loss of binding can exist in some neuroblastomas before treatment.

In particular, progressive disease during or after therapy with dinutuximab is due, at least in part, to resistance of NB to antibody therapy, the mechanisms of which are poorly understood. Most efforts towards elucidating the mechanisms of dinutuximab treatment failure in minimal residual disease have focused on the role of the effector cells involved in antibody-dependent cellular cytotoxicity. Studies seeking to overcome therapy resistance have largely focused on improving delivery of ancillary cytokines (such as with an immunocytokine) or approaches to enhance the activity of effector cells.

Moreover, prior literature has reported that most NBs at diagnosis express GD2 and that GD2-negativity in tumors recurring after GD2 therapy was infrequent. However, other studies demonstrated that low GD2-expressing NBs do occur, perhaps in as high as 12% of patients, in all stages of treatment and at relapse. That study also observed an increased frequency of low GD2-expression in NB patients at time of progressive disease. Another study demonstrated that a low % of GD2-positive neuroblastoma cells was associated with relapse in patients treated with dinutuximab.

Thus, low or negative GD2 expression may account for some treatment failures in NB patients treated with dinutuximab. Patients who do benefit from dinutuximab may be those with either a high cell surface density of GD2 expression and/or a high % of cells that bind dinutuximab.

As such, a need exists for more effective therapeutics for the treatment of cancers while circumventing cancer cell resistance. Numerous embodiments of the present disclosure aim to address the aforementioned need.

### Antibodies

In some embodiments, the present disclosure pertains to an antibody. In some embodiments, the antibody binds to GDNF family receptor alpha 2 (GFRA2). In some embodiments, the antibody is in a modified form. In some embodiments, the antibody is generated molecularly from the sequence of HSAN. In some embodiments, the molecularly generated antibody is in a modified form. In some embodiments, the modified form includes, without limitation, a chimeric form, a humanized form, a drug conjugated form, a bispecific form (e.g., an antibody combined with another antibody to recognize at least two different antigens), an immunocytokine form (e.g., an antibody fused to a cytokine), a labeled form, a fragmented form, or combinations thereof.

In some embodiments, the antibody includes a labeled antibody. In some embodiments, the labeled antibody includes a radioactive isotope label. In some embodiments, the labeled antibody includes a fluorescent label.

In some embodiments, the antibody is associated with a drug to form an antibody-drug conjugate. In some embodiments, the drug includes a cytotoxic agent. In some embodiments, the cytotoxic agent is operable to concentrate the cytotoxic agent at GFRA2-positive cells. In some embodiments, the GFRA2-positive cells include, without limitation, cancer cells, neuroblastoma cells, Ewing's sarcoma cells, thyroid cancer cells, or combinations thereof. In some embodiments, the drug includes an anti-cancer drug. In some embodiments, the anti-cancer drug includes, without limitation, pyrrolobenzodiazepine (PBD), monomethyl auristatin E (MMAE), deruxtecan (DXD), 7-ethyl-10-hydroxycamptothecin (SN38), duocarmycins, or combinations thereof.

The antibodies of the present disclosure may be in various forms. For instance, in some embodiments, the antibody includes, without limitation, a full-length antibody, a fragmented antibody, a Fab fragment, a humanized antibody, a chimeric antibody, a murine antibody, a monoclonal antibody, an isolated antibody, a bispecific antibody, an immunocytokine, or combinations thereof. In some embodiments, the antibody includes a humanized antibody. In some embodiments, the antibody includes a monoclonal antibody.

In some embodiments, the antibody includes a fragmented antibody. In some embodiments, the fragmented antibody includes a Fab fragment.

In some embodiments, the antibodies of the present disclosure may be suitable for use in treating or preventing a cancer in a subject. In some embodiments, the cancer includes, without limitation, GFRA2 positive cancers, neuroblastomas, Ewing's sarcoma, thyroid cancers, pediatric cancers, adult cancers, or combinations thereof. In some embodiments, the cancer includes neuroblastomas. In some embodiments, the cancer includes Ewing's sarcoma.

The antibodies of the present disclosure may have various sequences. For instance, in some embodiments, the antibodies of the present disclosure include one or more of the following complementarity determining regions (CDRs): SEQ ID NO: 5 or a sequence with at least 65%-99% sequence identity to SEQ ID NO: 5; SEQ ID NO: 6 or a sequence with at least 65%-99% sequence identity to SEQ ID NO: 6; SEQ ID NO: 7 or a sequence with at least 65%-99% sequence identity to SEQ ID NO: 7; SEQ ID NO: 8 or a sequence with at least 65%-99% sequence identity to SEQ ID NO: 8; SEQ ID NO: 9 or a sequence with at least 65%-99% sequence identity to SEQ ID NO: 9; SEQ ID NO: 10 or a sequence with at least 65%-99% sequence identity to SEQ ID NO: 10; SEQ ID NO: 11 or a sequence with at least 65%-99% sequence identity to SEQ ID NO: 11; and SEQ ID NO: 12 or a sequence with at least 65%-99% sequence identity to SEQ ID NO: 12.

In some embodiments, the antibodies of the present disclosure include one or more of the following CDRs: SEQ ID NO: 5 or a sequence with at least 65% sequence identity to SEQ ID NO: 5; SEQ ID NO: 6 or a sequence with at least 65% sequence identity to SEQ ID NO: 6; SEQ ID NO: 7 or a sequence with at least 65% sequence identity to SEQ ID NO: 7; SEQ ID NO: 8 or a sequence with at least 65% sequence identity to SEQ ID NO: 8; SEQ ID NO: 9 or a sequence with at least 65% sequence identity to SEQ ID NO: 9; SEQ ID NO: 10 or a sequence with at least 65% sequence identity to SEQ ID NO: 10; SEQ ID NO: 11 or a sequence with at least 65% sequence identity to SEQ ID NO: 11; and SEQ ID NO: 12 or a sequence with at least 65% sequence identity to SEQ ID NO: 12.

In some embodiments, the antibodies of the present disclosure include one or more of the following CDRs: SEQ ID NO: 5 or a sequence with at least 75% sequence identity to SEQ ID NO: 5; SEQ ID NO: 6 or a sequence with at least 75% sequence identity to SEQ ID NO: 6; SEQ ID NO: 7 or a sequence with at least 75% sequence identity to SEQ ID NO: 7; SEQ ID NO: 8 or a sequence with at least 75% sequence identity to SEQ ID NO: 8; SEQ ID NO: 9 or a sequence with at least 75% sequence identity to SEQ ID NO: 9; SEQ ID NO: 10 or a sequence with at least 75% sequence identity to SEQ ID NO: 10; SEQ ID NO: 11 or a sequence with at least 75% sequence identity to SEQ ID NO: 11; and SEQ ID NO: 12 or a sequence with at least 75% sequence identity to SEQ ID NO: 12.

In some embodiments, the antibodies of the present disclosure include one or more of the following CDRs: SEQ ID NO: 5 or a sequence with at least 85% sequence identity to SEQ ID NO: 5; SEQ ID NO: 6 or a sequence with at least 85% sequence identity to SEQ ID NO: 6; SEQ ID NO: 7 or a sequence with at least 85% sequence identity to SEQ ID NO: 7; SEQ ID NO: 8 or a sequence with at least 85% sequence identity to SEQ ID NO: 8; SEQ ID NO: 9 or a sequence with at least 85% sequence identity to SEQ ID NO: 9; SEQ ID NO: 10 or a sequence with at least 85% sequence identity to SEQ ID NO: 10; SEQ ID NO: 11 or a sequence with at least 85% sequence identity to SEQ ID NO: 11; and SEQ ID NO: 12 or a sequence with at least 85% sequence identity to SEQ ID NO: 12.

In some embodiments, the antibodies of the present disclosure include one or more of the following CDRs: SEQ ID NO: 5 or a sequence with at least 90% sequence identity to SEQ ID NO: 5; SEQ ID NO: 6 or a sequence with at least 90% sequence identity to SEQ ID NO: 6; SEQ ID NO: 7 or a sequence with at least 90% sequence identity to SEQ ID NO: 7; SEQ ID NO: 8 or a sequence with at least 90% sequence identity to SEQ ID NO: 8; SEQ ID NO: 9 or a sequence with at least 90% sequence identity to SEQ ID NO: 9; SEQ ID NO: 10 or a sequence with at least 90% sequence identity to SEQ ID NO: 10; SEQ ID NO: 11 or a sequence with at least 90% sequence identity to SEQ ID NO: 11; and SEQ ID NO: 12 or a sequence with at least 90% sequence identity to SEQ ID NO: 12.

In some embodiments, the antibodies of the present disclosure include one or more of the following CDRs: SEQ ID NO: 5 or a sequence with at least 95% sequence identity to SEQ ID NO: 5; SEQ ID NO: 6 or a sequence with at least 95% sequence identity to SEQ ID NO: 6; SEQ ID NO: 7 or a sequence with at least 95% sequence identity to SEQ ID NO: 7; SEQ ID NO: 8 or a sequence with at least 95% sequence identity to SEQ ID NO: 8; SEQ ID NO: 9 or a sequence with at least 95% sequence identity to SEQ ID NO: 9; SEQ ID NO: 10 or a sequence with at least 95% sequence identity to SEQ ID NO: 10; SEQ ID NO: 11 or a sequence with at least 95% sequence identity to SEQ ID NO: 11; and SEQ ID NO: 12 or a sequence with at least 95% sequence identity to SEQ ID NO: 12.

In some embodiments, the antibodies of the present disclosure include one or more of the following CDRs: SEQ ID NO: 5 or a sequence with at least 99% sequence identity to SEQ ID NO: 5; SEQ ID NO: 6 or a sequence with at least 99% sequence identity to SEQ ID NO: 6; SEQ ID NO: 7 or a sequence with at least 99% sequence identity to SEQ ID NO: 7; SEQ ID NO: 8 or a sequence with at least 99% sequence identity to SEQ ID NO: 8; SEQ ID NO: 9 or a sequence with at least 99% sequence identity to SEQ ID NO: 9; SEQ ID NO: 10 or a sequence with at least 99% sequence identity to SEQ ID NO: 10; SEQ ID NO: 11 or a sequence with at least 99% sequence identity to SEQ ID NO: 11; and SEQ ID NO: 12 or a sequence with at least 99% sequence identity to SEQ ID NO: 12.

In some embodiments, the antibodies of the present disclosure include the following CDRs: SEQ ID NO: 5; SEQ ID NO: 6; SEQ ID NO: 7; SEQ ID NO: 8; SEQ ID NO: 9; and SEQ ID NO: 10. In some embodiments, SEQ ID NOs: 5-7 represent light chain CDR 1, CDR2, and CDR3, respectively. In some embodiments, SEQ ID NOs: 8-10 represent heavy chain CDR1, CDR2, and CDR3, respectively.

In some embodiments, the antibodies of the present disclosure include the following CDRs: SEQ ID NO: 5; SEQ ID NO: 6; SEQ ID NO: 7; SEQ ID NO: 10; SEQ ID NO: 11; and SEQ ID NO: 12. In some embodiments, SEQ ID NOs: 5-7 represent light chain CDR 1, CDR2, and CDR3, respectively. In some embodiments, SEQ ID NOs: 11, 12, and 10 represent heavy chain CDR1, CDR2, and CDR3, respectively.

In some embodiments, the antibody light chain includes SEQ ID NO: 1 or a sequence with at least 50% sequence identity with SEQ ID NO: 1. In some embodiments, the antibody light chain includes SEQ ID NO: 1 or a sequence with at least 65% sequence identity with SEQ ID NO: 1. In some embodiments, the antibody light chain includes SEQ ID NO: 1 or a sequence with at least 75% sequence identity with SEQ ID NO: 1. In some embodiments, the antibody light chain includes SEQ ID NO: 1 or a sequence with at least 85% sequence identity with SEQ ID NO: 1. In some embodiments, the antibody light chain includes SEQ ID NO: 1 or a sequence with at least 90% sequence identity with SEQ ID NO: 1. In some embodiments, the antibody light chain includes SEQ ID NO: 1 or a sequence with at least 95% sequence identity with SEQ ID NO: 1. In some embodiments, the antibody light chain includes SEQ ID NO: 1 or a sequence with at least 99% sequence identity with SEQ ID NO: 1. In some embodiments, the antibody light chain includes SEQ ID NO: 1.

In some embodiments, the antibody light chain includes SEQ ID NO: 4 or a sequence with at least 50% sequence identity with SEQ ID NO: 4. In some embodiments, the antibody light chain includes SEQ ID NO: 4 or a sequence with at least 65% sequence identity with SEQ ID NO: 4. In some embodiments, the antibody light chain includes SEQ ID NO: 4 or a sequence with at least 75% sequence identity with SEQ ID NO: 4. In some embodiments, the antibody light chain includes SEQ ID NO: 4 or a sequence with at least 85% sequence identity with SEQ ID NO: 4. In some embodiments, the antibody light chain includes SEQ ID NO: 4 or a sequence with at least 90% sequence identity with SEQ ID NO: 4. In some embodiments, the antibody light chain includes SEQ ID NO: 4 or a sequence with at least 95% sequence identity with SEQ ID NO: 4. In some embodiments, the antibody light chain includes SEQ ID NO: 4 or a sequence with at least 99% sequence identity with SEQ ID NO: 4. In some embodiments, the antibody light chain includes SEQ ID NO: 4.

In some embodiments, the antibody heavy chain includes SEQ ID NO: 2 or a sequence with at least 50% sequence identity with SEQ ID NO: 2. In some embodiments, the antibody heavy chain includes SEQ ID NO: 2 or a sequence with at least 65% sequence identity with SEQ ID NO: 2. In some embodiments, the antibody heavy chain includes SEQ ID NO: 2 or a sequence with at least 75% sequence identity with SEQ ID NO: 2. In some embodiments, the antibody heavy chain includes SEQ ID NO: 2 or a sequence with at least 85% sequence identity with SEQ ID NO: 2. In some embodiments, the antibody heavy chain includes SEQ ID NO: 2 or a sequence with at least 90% sequence identity with SEQ ID NO: 2. In some embodiments, the antibody heavy chain includes SEQ ID NO: 2 or a sequence with at least 95% sequence identity with SEQ ID NO: 2. In some embodiments, the antibody heavy chain includes SEQ ID NO: 2 or a sequence with at least 99% sequence identity with SEQ ID NO: 2. In some embodiments, the antibody heavy chain includes SEQ ID NO: 2.

In some embodiments, the antibody heavy chain includes SEQ ID NO: 3 or a sequence with at least 50% sequence identity with SEQ ID NO: 3. In some embodiments, the antibody heavy chain includes SEQ ID NO: 3 or a sequence with at least 65% sequence identity with SEQ ID NO: 3. In some embodiments, the antibody heavy chain includes SEQ ID NO: 3 or a sequence with at least 75% sequence identity with SEQ ID NO: 3. In some embodiments, the antibody heavy chain includes SEQ ID NO: 3 or a sequence with at least 85% sequence identity with SEQ ID NO: 3. In some embodiments, the antibody heavy chain includes SEQ ID NO: 3 or a sequence with at least 90% sequence identity with SEQ ID NO: 3. In some embodiments, the antibody heavy chain includes SEQ ID NO: 3 or a sequence with at least 95% sequence identity with SEQ ID NO: 3. In some embodiments, the antibody heavy chain includes SEQ ID NO: 3 or a sequence with at least 99% sequence identity with SEQ ID NO: 3. In some embodiments, the antibody heavy chain includes SEQ ID NO: 3.

### Humanized Antibodies

In some embodiments, the antibodies of the present disclosure include humanized antibodies. In some embodiments, the humanized antibodies include humanized monoclonal antibodies.

The humanized antibodies of the present disclosure may have various sequences. For instance, in some embodiments, the antibodies of the present disclosure include one or more of the following CDRs: KSSQSLLDTDGKTYLN (SEQ ID NO: 15) or a sequence with at least 65%-99% sequence identity to SEQ ID NO: 15; LVSKLDS (SEQ ID NO: 16) or a sequence with at least 65%-99% sequence identity to SEQ ID NO: 16; WQGTHFPQT (SEQ ID NO: 17) or a sequence with at least 65%-99% sequence identity to SEQ ID NO: 17; GYFMH (SEQ ID NO: 18) or a sequence with at least 65%-99% sequence identity to SEQ ID NO: 18; RINPYNGATN YNQIFKD (SEQ ID NO: 19) or a sequence with at least 65%-99% sequence identity to SEQ ID NO: 19; GGHYYGNVDY (SEQ ID NO: 20) or a sequence with at least 65%-99% sequence identity to SEQ ID NO: 20; GYSFTGY (SEQ ID NO: 21) or a sequence with at least 65%-99% sequence identity to SEQ ID NO: 21; and NPYNGA (SEQ ID NO: 22) or a sequence with at least 65%-99% sequence identity to SEQ ID NO: 22.

In some embodiments, the antibodies of the present disclosure include one or more of the following CDRs: SEQ ID NO: 15 or a sequence with at least 65% sequence identity to SEQ ID NO: 15; SEQ ID NO: 16 or a sequence with at least 65% sequence identity to SEQ ID NO: 16; SEQ ID NO: 17 or a sequence with at least 65% sequence identity to SEQ ID NO: 17; SEQ ID NO: 18 or a sequence with at least 65% sequence identity to SEQ ID NO: 18; SEQ ID NO: 19 or a sequence with at least 65% sequence identity to SEQ ID NO: 19; SEQ ID NO: 20 or a sequence with at least 65% sequence identity to SEQ ID NO: 20; SEQ ID NO: 21 or a sequence with at least 65% sequence identity to SEQ ID NO: 21; and SEQ ID NO: 22 or a sequence with at least 65% sequence identity to SEQ ID NO: 22.

In some embodiments, the antibodies of the present disclosure include one or more of the following CDRs: SEQ ID NO: 15 or a sequence with at least 75% sequence identity to SEQ ID NO: 15; SEQ ID NO: 16 or a sequence with at least 75% sequence identity to SEQ ID NO: 16; SEQ ID NO: 17 or a sequence with at least 75% sequence identity to SEQ ID NO: 17; SEQ ID NO: 18 or a sequence with at least 75% sequence identity to SEQ ID NO: 18; SEQ ID NO: 19 or a sequence with at least 75% sequence identity to SEQ ID NO: 19; SEQ ID NO: 20 or a sequence with at least 75% sequence identity to SEQ ID NO: 20; SEQ ID NO: 21 or a sequence with at least 75% sequence identity to SEQ ID NO: 21; and SEQ ID NO: 22 or a sequence with at least 75% sequence identity to SEQ ID NO: 22.

In some embodiments, the antibodies of the present disclosure include one or more of the following CDRs: SEQ ID NO: 15 or a sequence with at least 85% sequence identity to SEQ ID NO: 15; SEQ ID NO: 16 or a sequence with at least 85% sequence identity to SEQ ID NO: 16; SEQ ID NO: 17 or a sequence with at least 85% sequence identity to SEQ ID NO: 17; SEQ ID NO: 18 or a sequence with at least 85% sequence identity to SEQ ID NO: 18; SEQ ID NO: 19 or a sequence with at least 85% sequence identity to SEQ ID NO: 19; SEQ ID NO: 20 or a sequence with at least 85% sequence identity to SEQ ID NO: 20; SEQ ID NO: 21 or a sequence with at least 85% sequence identity to SEQ ID NO: 21; and SEQ ID NO: 22 or a sequence with at least 85% sequence identity to SEQ ID NO: 22.

In some embodiments, the antibodies of the present disclosure include one or more of the following CDRs: SEQ ID NO: 15 or a sequence with at least 90% sequence identity to SEQ ID NO: 15; SEQ ID NO: 16 or a sequence with at least 90% sequence identity to SEQ ID NO: 16; SEQ ID NO: 17 or a sequence with at least 90% sequence identity to SEQ ID NO: 17; SEQ ID NO: 18 or a sequence with at least 90% sequence identity to SEQ ID NO: 18; SEQ ID NO: 19 or a sequence with at least 90% sequence identity to SEQ ID NO: 19; SEQ ID NO: 20 or a sequence with at least 90% sequence identity to SEQ ID NO: 20; SEQ ID NO: 21 or a sequence with at least 90% sequence identity to SEQ ID NO: 21; and SEQ ID NO: 22 or a sequence with at least 90% sequence identity to SEQ ID NO: 22.

In some embodiments, the antibodies of the present disclosure include one or more of the following CDRs: SEQ ID NO: 15 or a sequence with at least 95% sequence identity to SEQ ID NO: 15; SEQ ID NO: 16 or a sequence with at least 95% sequence identity to SEQ ID NO: 16; SEQ ID NO: 17 or a sequence with at least 95% sequence identity to SEQ ID NO: 17; SEQ ID NO: 18 or a sequence with at least 95% sequence identity to SEQ ID NO: 18; SEQ ID NO: 19 or a sequence with at least 95% sequence identity to SEQ ID NO: 19; SEQ ID NO: 20 or a sequence with at least 95% sequence identity to SEQ ID NO: 20; SEQ ID NO: 21 or a sequence with at least 95% sequence identity to SEQ ID NO: 21; and SEQ ID NO: 22 or a sequence with at least 95% sequence identity to SEQ ID NO: 22.

In some embodiments, the antibodies of the present disclosure include one or more of the following CDRs: SEQ ID NO: 15 or a sequence with at least 99% sequence identity to SEQ ID NO: 15; SEQ ID NO: 16 or a sequence with at least 99% sequence identity to SEQ ID NO: 16; SEQ ID NO: 17 or a sequence with at least 99% sequence identity to SEQ ID NO: 17; SEQ ID NO: 18 or a sequence with at least 99% sequence identity to SEQ ID NO: 18; SEQ ID NO: 19 or a sequence with at least 99% sequence identity to SEQ ID NO: 19; SEQ ID NO: 20 or a sequence with at least 99% sequence identity to SEQ ID NO: 20; SEQ ID NO: 21 or a sequence with at least 99% sequence identity to SEQ ID NO: 21; and SEQ ID NO: 22 or a sequence with at least 99% sequence identity to SEQ ID NO: 22.

In some embodiments, the antibodies of the present disclosure include one or more of the following CDRs: SEQ ID NO: 15; SEQ ID NO: 16; SEQ ID NO: 17; SEQ ID NO: 18; SEQ ID NO: 19; SEQ ID NO: 20; SEQ ID NO: 21; and SEQ ID NO: 22.

In some embodiments, the antibodies of the present disclosure include the following CDRs: SEQ ID NO: 15; SEQ ID NO: 16; SEQ ID NO: 17; SEQ ID NO: 18; SEQ ID NO: 19; and SEQ ID NO: 20. In some embodiments, SEQ ID NOs: 15-17 represent light chain CDR 1, CDR2, and CDR3, respectively. In some embodiments, SEQ ID NOs: 18-20 represent heavy chain CDR1, CDR2, and CDR3, respectively.

In some embodiments, the antibodies of the present disclosure include the following CDRs: SEQ ID NO: 15; SEQ ID NO: 16; SEQ ID NO: 17; SEQ ID NO: 19; SEQ ID NO: 20; and SEQ ID NO: 21. In some embodiments, SEQ ID NOs: 15-17 represent light chain CDR 1, CDR2, and CDR3, respectively. In some embodiments, SEQ ID NOs: 19, 20, and 21 represent heavy chain CDR2, CDR3, and CDR1, respectively.

In some embodiments, the antibodies of the present disclosure include the following CDRs: SEQ ID NO: 15; SEQ ID NO: 16; SEQ ID NO: 17; SEQ ID NO: 20; SEQ ID NO: 21; and SEQ ID NO: 22. In some embodiments, SEQ ID NOs: 15-17 represent light chain CDR 1, CDR2, and CDR3, respectively. In some embodiments, SEQ ID NOs: 21, 22, and 20 represent heavy chain CDR1, CDR2, and CDR3, respectively.

The antibodies of the present disclosure can include various full length sequences. For instance, in some embodiments, the full length sequence includes SEQ ID NO: 23 or a sequence with at least 65% sequence identity to SEQ ID NO: 23. In some embodiments, the full length sequence includes SEQ ID NO: 23 or a sequence with at least 75% sequence identity to SEQ ID NO: 23. In some embodiments, the full length sequence includes SEQ ID NO: 23 or a sequence with at least 85% sequence identity to SEQ ID NO: 23. In some embodiments, the full length sequence includes SEQ ID NO: 23 or a sequence with at least 90% sequence identity to SEQ ID NO: 23. In some embodiments, the full length sequence includes SEQ ID NO: 23 or a sequence with at least 95% sequence identity to SEQ ID NO: 23. In some embodiments, the full length sequence includes SEQ ID NO: 23 or a sequence with at least 99% sequence identity to SEQ ID NO: 23. In some embodiments, the full length sequence includes SEQ ID NO: 23.

In some embodiments, the full length sequence includes SEQ ID NO: 24 or a sequence with at least 65% sequence identity to SEQ ID NO: 24. In some embodiments, the full length sequence includes SEQ ID NO: 24 or a sequence with at least 75% sequence identity to SEQ ID NO: 24. In some embodiments, the full length sequence includes SEQ ID NO: 24 or a sequence with at least 85% sequence identity to SEQ ID NO: 24. In some embodiments, the full length sequence includes SEQ ID NO: 24 or a sequence with at least 90% sequence identity to SEQ ID NO: 24. In some embodiments, the full length sequence includes SEQ ID NO: 24 or a sequence with at least 95% sequence identity to SEQ ID NO: 24. In some embodiments, the full length sequence includes SEQ ID NO: 24 or a sequence with at least 99% sequence identity to SEQ ID NO: 24. In some embodiments, the full length sequence includes SEQ ID NO: 24.

In some embodiments, the full length sequence includes SEQ ID NO: 25 or a sequence with at least 65% sequence identity to SEQ ID NO: 25. In some embodiments, the full length sequence includes SEQ ID NO: 25 or a sequence with at least 75% sequence identity to SEQ ID NO: 25. In some embodiments, the full length sequence includes SEQ ID NO: 25 or a sequence with at least 85% sequence identity to SEQ ID NO: 25. In some embodiments, the full length sequence includes SEQ ID NO: 25 or a sequence with at least 90% sequence identity to SEQ ID NO: 25. In some embodiments, the full length sequence includes SEQ ID NO: 25 or a sequence with at least 95% sequence identity to SEQ ID NO: 25. In some embodiments, the full length sequence includes SEQ ID NO: 25 or a sequence with at least 99% sequence identity to SEQ ID NO: 25. In some embodiments, the full length sequence includes SEQ ID NO: 25.

In some embodiments, the full length sequence includes SEQ ID NO: 26 or a sequence with at least 65% sequence identity to SEQ ID NO: 26. In some embodiments, the full length sequence includes SEQ ID NO: 26 or a sequence with at least 75% sequence identity to SEQ ID NO: 26. In some embodiments, the full length sequence includes SEQ ID NO: 26 or a sequence with at least 85% sequence identity to SEQ ID NO: 26. In some embodiments, the full length sequence includes SEQ ID NO: 26 or a sequence with at least 90% sequence identity to SEQ ID NO: 26. In some embodiments, the full length sequence includes SEQ ID NO: 26 or a sequence with at least 95% sequence identity to SEQ ID NO: 26. In some embodiments, the full length sequence includes SEQ ID NO: 26 or a sequence with at least 99% sequence identity to SEQ ID NO: 26. In some embodiments, the full length sequence includes SEQ ID NO: 26.

In some embodiments, the full length sequence includes SEQ ID NO: 27 or a sequence with at least 65% sequence identity to SEQ ID NO: 27. In some embodiments, the full length sequence includes SEQ ID NO: 27 or a sequence with at least 75% sequence identity to SEQ ID NO: 27. In some embodiments, the full length sequence includes SEQ ID NO: 27 or a sequence with at least 85% sequence identity to SEQ ID NO: 27. In some embodiments, the full length sequence includes SEQ ID NO: 27 or a sequence with at least 90% sequence identity to SEQ ID NO: 27. In some embodiments, the full length sequence includes SEQ ID NO: 27 or a sequence with at least 95% sequence identity to SEQ ID NO: 27. In some embodiments, the full length sequence includes SEQ ID NO: 27 or a sequence with at least 99% sequence identity to SEQ ID NO: 27. In some embodiments, the full length sequence includes SEQ ID NO: 27.

In some embodiments, the full length sequence includes SEQ ID NO: 28 or a sequence with at least 65% sequence identity to SEQ ID NO: 28. In some embodiments, the full length sequence includes SEQ ID NO: 28 or a sequence with at least 75% sequence identity to SEQ ID NO: 28. In some embodiments, the full length sequence includes SEQ ID NO: 28 or a sequence with at least 85% sequence identity to SEQ ID NO: 28. In some embodiments, the full length sequence includes SEQ ID NO: 28 or a sequence with at least 90% sequence identity to SEQ ID NO: 28. In some embodiments, the full length sequence includes SEQ ID NO: 28 or a sequence with at least 95% sequence identity to SEQ ID NO: 28. In some embodiments, the full length sequence includes SEQ ID NO: 28 or a sequence with at least 99% sequence identity to SEQ ID NO: 28. In some embodiments, the full length sequence includes SEQ ID NO: 28.

In some embodiments, the full length sequence includes SEQ ID NO: 29 or a sequence with at least 65% sequence identity to SEQ ID NO: 29. In some embodiments, the full length sequence includes SEQ ID NO: 29 or a sequence with at least 75% sequence identity to SEQ ID NO: 29. In some embodiments, the full length sequence includes SEQ ID NO: 29 or a sequence with at least 85% sequence identity to SEQ ID NO: 29. In some embodiments, the full length sequence includes SEQ ID NO: 29 or a sequence with at least 90% sequence identity to SEQ ID NO: 29. In some embodiments, the full length sequence includes SEQ ID NO: 29 or a sequence with at least 95% sequence identity to SEQ ID NO: 29. In some embodiments, the full length sequence includes SEQ ID NO: 29 or a sequence with at least 99% sequence identity to SEQ ID NO: 29. In some embodiments, the full length sequence includes SEQ ID NO: 29.

In some embodiments, the full length sequence includes SEQ ID NO: 30 or a sequence with at least 65% sequence identity to SEQ ID NO: 30. In some embodiments, the full length sequence includes SEQ ID NO: 30 or a sequence with at least 75% sequence identity to SEQ ID NO: 30. In some embodiments, the full length sequence includes SEQ ID NO: 30 or a sequence with at least 85% sequence identity to SEQ ID NO: 30. In some embodiments, the full length sequence includes SEQ ID NO: 30 or a sequence with at least 90% sequence identity to SEQ ID NO: 30. In some embodiments, the full length sequence includes SEQ ID NO: 30 or a sequence with at least 95% sequence identity to SEQ ID NO: 30. In some embodiments, the full length sequence includes SEQ ID NO: 30 or a sequence with at least 99% sequence identity to SEQ ID NO: 30. In some embodiments, the full length sequence includes SEQ ID NO: 30.

In some embodiments, the full length sequence includes SEQ ID NO: 31 or a sequence with at least 65% sequence identity to SEQ ID NO: 31. In some embodiments, the full length sequence includes SEQ ID NO: 31 or a sequence with at least 75% sequence identity to SEQ ID NO: 31. In some embodiments, the full length sequence includes SEQ ID NO: 31 or a sequence with at least 85% sequence identity to SEQ ID NO: 31. In some embodiments, the full length sequence includes SEQ ID NO: 31 or a sequence with at least 90% sequence identity to SEQ ID NO: 31. In some embodiments, the full length sequence includes SEQ ID NO: 31 or a sequence with at least 95% sequence identity to SEQ ID NO: 31. In some embodiments, the full length sequence includes SEQ ID NO: 31 or a sequence with at least 99% sequence identity to SEQ ID NO: 31. In some embodiments, the full length sequence includes SEQ ID NO: 31.

In some embodiments, the full length sequence includes SEQ ID NO: 32 or a sequence with at least 65% sequence identity to SEQ ID NO: 32. In some embodiments, the full length sequence includes SEQ ID NO: 32 or a sequence with at least 75% sequence identity to SEQ ID NO: 32. In some embodiments, the full length sequence includes SEQ ID NO: 32 or a sequence with at least 85% sequence identity to SEQ ID NO: 32. In some embodiments, the full length sequence includes SEQ ID NO: 32 or a sequence with at least 90% sequence identity to SEQ ID NO: 32. In some embodiments, the full length sequence includes SEQ ID NO: 32 or a sequence with at least 95% sequence identity to SEQ ID NO: 32. In some embodiments, the full length sequence includes SEQ ID NO: 32 or a sequence with at least 99% sequence identity to SEQ ID NO: 32. In some embodiments, the full length sequence includes SEQ ID NO: 32.

### Methods of Using Antibodies to Treat or Prevent Cancer in a Subject

Additional embodiments of the present disclosure pertain to methods of treating or preventing a cancer in a subject by administering to the subject an antibody of the present disclosure. As set forth in more detail herein, the antibody treatment methods of the present disclosure can have numerous embodiments.

The antibody treatment methods of the present disclosure may utilize various antibodies of the present disclosure. Such antibodies were described supra and are incorporated herein by reference.

Various methods may be utilized to administer the antibodies of the present disclosure to subjects. For instance, in some embodiments, the administration occurs by a method that includes, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, local administration, or combinations thereof. In some embodiments, the administration includes local administration to a specific tissue of a subject. In some embodiments, the tissue includes a tumor.

The antibody treatment methods of the present disclosure may be utilized to treat various subjects. For instance, in some embodiments, the subject is a human being. In some embodiments, the subject is a pediatric patient.

In some embodiments, the subject is suffering from cancer. As such, in some embodiments, the methods of the present disclosure are utilized to treat the cancer. In some embodiments, the subject is vulnerable to cancer. As such, in some embodiments, the methods of the present disclosure may be utilized to prevent the cancer.

The antibody treatment methods of the present disclosure may be utilized to treat or prevent various cancers. For instance, in some embodiments, the cancer to be treated or prevented includes, without limitation, GFRA2 positive cancers, neuroblastomas, Ewing's sarcoma, thyroid cancers, pediatric cancers, adult cancers, or combinations thereof. In some embodiments, the cancer to be treated or prevented includes pediatric cancers. In some embodiments, the cancer to be treated or prevented includes neuroblastomas. In some embodiments, the cancer to be treated or prevented includes Ewing's sarcoma.

The antibodies of the present disclosure may be administered to a subject at various frequencies sufficient to treat or prevent a cancer in the subject. For instance, in some embodiments, the antibodies of the present disclosure may be administered one or more times per day, once daily, once every two days, once every three days, once weekly, once every two weeks, once every three weeks, once monthly, or at another clinically appropriate interval. In some embodiments, an initial loading dose or loading regimen is administered, followed by one or more maintenance doses.

In some embodiments, the frequency of antibody administration is adjusted based on one or more factors. Such factors may include, without limitation, the type of cancer, disease burden, stage of disease, age of the subject, body weight, route of administration, observed response, tolerability, pharmacokinetic profile, or combinations thereof. In some embodiments, a frequency of administration may be selected to maintain a therapeutically effective amount of an antibody of the present disclosure in the subject over a desired treatment period.

The antibodies of the present disclosure may be administered to a subject at various amounts sufficient to treat or prevent a cancer in the subject. For instance, in some embodiments, the amount administered may be expressed as a fixed dose or may be based on a subject parameter. In some embodiments, the parameters may include, without limitation, body weight, body surface area, or combinations thereof. In some embodiments, the antibodies of the present disclosure may be administered at a dose of about 0.01 mg/kg to about 100 mg/kg, about 0.1 mg/kg to about 50 mg/kg, or about 1 mg/kg to about 20 mg/kg. In some embodiments, the administered dose is about 1 mg/kg, about 3 mg/kg, about 5 mg/kg, about 10 mg/kg, or about 15 mg/kg.

In some embodiments, the amount of antibody administered may be adjusted over the course of treatment based on one or more factors. In some embodiments, such factors may include, without limitation, therapeutic response, tolerability, route of administration, dosing frequency, or combinations thereof. In some embodiments, the amount of antibody administered is sufficient to reduce tumor burden, inhibit tumor growth, delay disease progression, or otherwise provide a therapeutic benefit to the subject.

Methods of Using Antibodies to Diagnose a Subject with a GRFA2 Positive Cancer

In additional embodiments, the present disclosure pertains to methods of diagnosing a subject with a GRFA2 positive cancer. In some embodiments, the methods of the present disclosure include: exposing a specimen from the subject to an antibody of the present disclosure; detecting binding of the antibody to the specimen; and correlating the binding to the presence of the GRFA2 positive cancer in the subject.

The detection methods of the present disclosure may utilize various antibodies of the present disclosure. Such antibodies were described supra and are incorporated herein by reference.

The detection methods of the present disclosure can occur in various settings. For instance, in some embodiments, the detecting occurs in vivo in the subject. As such, in some embodiments, the methods of the present disclosure may be utilized to locate the cancer in the subject.

In some embodiments, the detecting occurs in vitro. In some embodiments, the methods of the present disclosure also include a step of collecting a specimen from the subject. In some embodiments, the specimen includes, without limitation, cells, blood, tissues, tumors, bone marrow, or combinations thereof.

Various methods may be utilized to detect antibody binding to specimen. For instance, in some embodiments, the detecting occurs by a method that includes, without limitation, flow cytometry, fluorescence microscopy, light microscopy, imaging, or combinations thereof. In some embodiments, the antibody is conjugated to a fluorescent dye, a metal isotope, or a radioactive isotope for the detection.

In some embodiments, the diagnostic methods of the present disclosure may be utilized to detect tumor cells in various specimen, such as blood and/or bone marrow. In some embodiments, the diagnostic methods of the present disclosure may be utilized to identify neuroblastoma cells in mixtures of other cell types. In some of such embodiments, such methods may be utilized to assess binding of anti-GD2 antibodies to the neuroblastoma cells.

In some embodiments, the methods of the present disclosure also include a step of implementing a treatment decision based on the detecting. In some embodiments, the treatment decision includes monitoring the subject for signs or symptoms of the cancer, administering a therapeutic agent to the subject, removal of GRFA2 positive cancer cells from the subject, or combinations thereof. In some embodiments, the treatment decision includes removal of GRFA2 positive cancer cells from the bone marrow of the subject. In some embodiments, the method is repeated after implementing the treatment decision.

The methods of the present disclosure can be utilized to diagnose subjects with various types of GFRA2 positive cancers. For instance, in some embodiments, the cancer includes, without limitation, neuroblastomas, Ewing's sarcoma, thyroid cancer, pediatric cancers, adult cancers, or combinations thereof.

### CAR T-Cells

Additional embodiments of the present disclosure pertain to chimeric antigen receptor T-cells (CAR T-cell) that express an antibody of the present disclosure. The CAR T-cells of the present disclosure may express various antibodies of the present disclosure. Such antibodies were described supra and are incorporated herein by reference. For instance, in some embodiments, the expressed antibodies include an antibody fragment, such as a Fab fragment.

In some embodiments, the CAR T-cells of the present disclosure express the antibodies of the present disclosure on a surface of the cells such that the antibodies are able to bind to GFRA2 positive cancer cells. In some embodiments, such binding facilitates the killing of the cancer cells by the CAR T-cells.

The CAR T-cells of the present disclosure may be in various forms. For instance, in some embodiments, the CAR T-cells of the present disclosure include T lymphocytes, natural killer (NK) cells, natural killer T (NKT) cells, gamma delta T cells, memory T cells, effector T cells, stem cell memory T cells, or combinations thereof. In some embodiments, the CAR T-cells of the present disclosure include chimeric antigen receptor natural killer cells (CAR-NKT).

In some embodiments, the CAR T-cells of the present disclosure are suitable for use in treating or preventing a cancer in a subject. In some embodiments, the cancer includes, without limitation, GFRA2 positive cancers, neuroblastomas, Ewing's sarcoma, pediatric cancers, or combinations thereof.

### Methods of Using CAR T-Cells to Treat or Prevent Cancer in a Subject

Additional embodiments of the present disclosure pertain to methods of treating or preventing a cancer in a subject by administering to the subject a CAR T-cell of the present disclosure. The methods of the present disclosure may utilize various CAR T-cells to treat or prevent a cancer in a subject. Suitable CAR T-cells were described supra and are incorporated herein by reference.

Various methods may be utilized to administer the CAR T-cells of the present disclosure to a subject. For instance, in some embodiments, the administering occurs by a method that includes, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, local administration, or combinations thereof. In some embodiments, the administering includes local administration to a specific tissue of a subject. In some embodiments, the tissue includes a tumor.

The CAR T-cell treatment methods of the present disclosure may be utilized to treat various subjects. For instance, in some embodiments, the subject is a human being. In some embodiments, the subject is a pediatric patient.

In some embodiments, the subject is suffering from cancer. As such, in some embodiments, the method is utilized to treat the cancer. In some embodiments, the subject is vulnerable to cancer. As such, in some embodiments, the method is utilized to prevent the cancer.

The CAR T-cell treatment methods of the present disclosure may be utilized to treat or prevent various cancers. For instance, in some embodiments, the cancer to be treated or prevented includes, without limitation, GFRA2 positive cancers, neuroblastomas, Ewing's sarcoma, thyroid cancer, pediatric cancers, adult cancers, or combinations thereof. In some embodiments, the cancer to be treated or prevented includes pediatric cancers.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Development, Characterization, and Use of HSAN Anti-Neuroblastoma Monoclonal Antibodies

Previously, Applicant developed an antibody that was highly specific for neuroblastoma (i.e., an HSAN antibody). However, defining the antigen HSAN binds to remained elusive. The HSAN antibody has been used for purging of neuroblastoma cells from bone marrow to enable autologous bone marrow transplantation of neuroblastoma and has been used for detection of neuroblastoma cells in bone marrow.

In this Example, Applicant aimed to determine if loss of the GD2 antigen for dinutuximab, an antibody currently used for and FDA approved for treating neuroblastoma, could result in therapeutic resistance. In this Example, Applicant also aimed to determine if HSAN could be utilized as an alternative antibody for treating neuroblastoma (NB).

### Example 1.1. Assessing GD2 Expression (Dinutuximab Binding) in NB Patients with Progressive Disease

Although increasing in frequency due to ‘basket’ trials evaluating targeted agents, serial tumor biopsy in children with progressive NB is not commonly performed. In contrast, bone marrow aspirates are usually obtained from NB patients at diagnosis and at time of progressive disease. Frequently, marrow aspirates from NB patients at time of progressive disease have tumor cells, but tumor cell number can be low.

A study on GD2 expression took advantage of the common availability of bone marrow aspirates to assess GD2 positivity in NB. While that study demonstrated the potential for low or negative GD2 expression in a subset of NB patients, there were limitations to the study in that it utilized immunohistochemistry for detection with tumor cell identification by morphology only-quantitation of GD2 expression per NB cell was not assessed. Thus, it is desirable to measure GD2 antigen expression in progressive NB using methods capable of quantitatively assessing both relative GD2 single tumor cell surface expression and the fraction of total tumor cells positive vs. negative for GD2.

Another limitation of the study was the sole reliance on cell morphology to determine if GD2-negative NB cells were present in the marrow. By contrast, multi-color fluorescence detection, quantified by flow cytometry, enables an assessment of both overall GD2 positivity and a robust quantification of the GD2 expression of individual NB cells in the aspirate (when NB cells are detected using NB-specific antibodies other than anti-GD2). Therefore, Applicant has employed both fluorescently-labeled dinutuximab, and a NB-specific, non-GD2-binding antibody, HSAN which does not cross-react with normal blood or marrow cells.

### Example 1.2. Quantifying GD2 Expression in a Panel of NB Patient-Derived Cell Lines (PDCLs) and Patient-Derived Xenografts (PDXs)

Applicant has labeled dinutuximab with the fluorophore, Alexa Fluor 647, to enable accurate quantification of dinutuximab NB cell binding by flow cytometry (BD LSRFortessa flow cytometer).

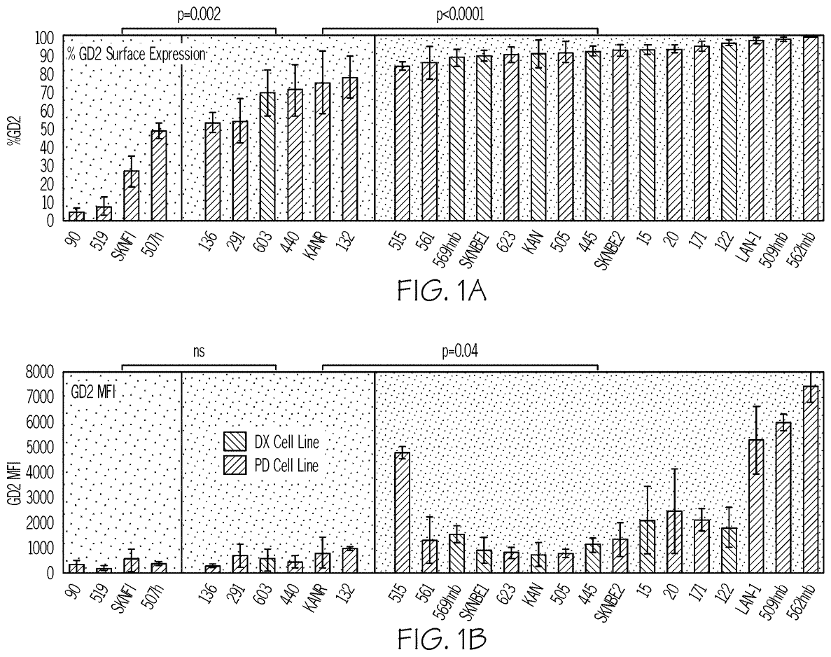

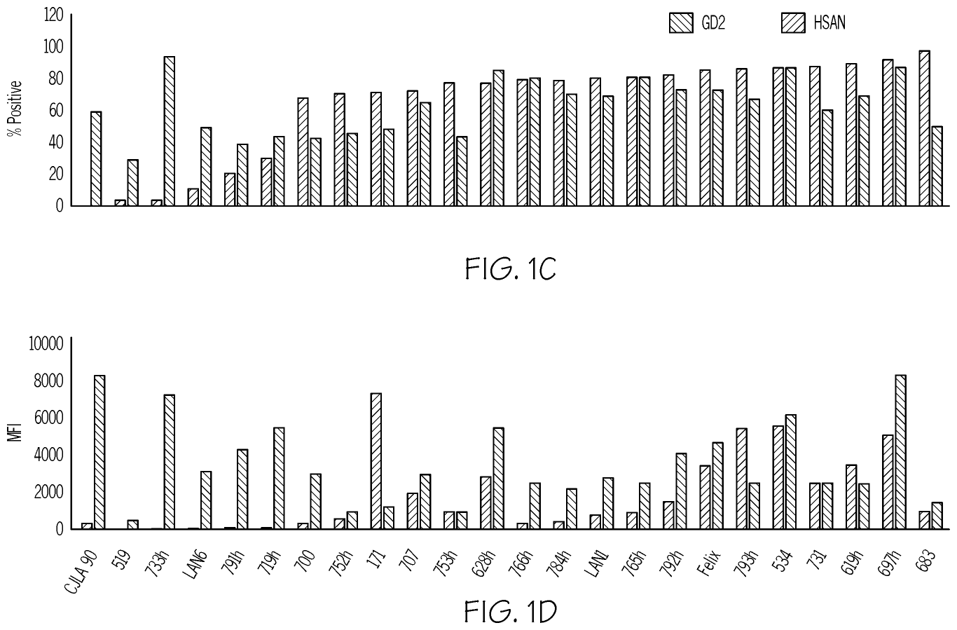

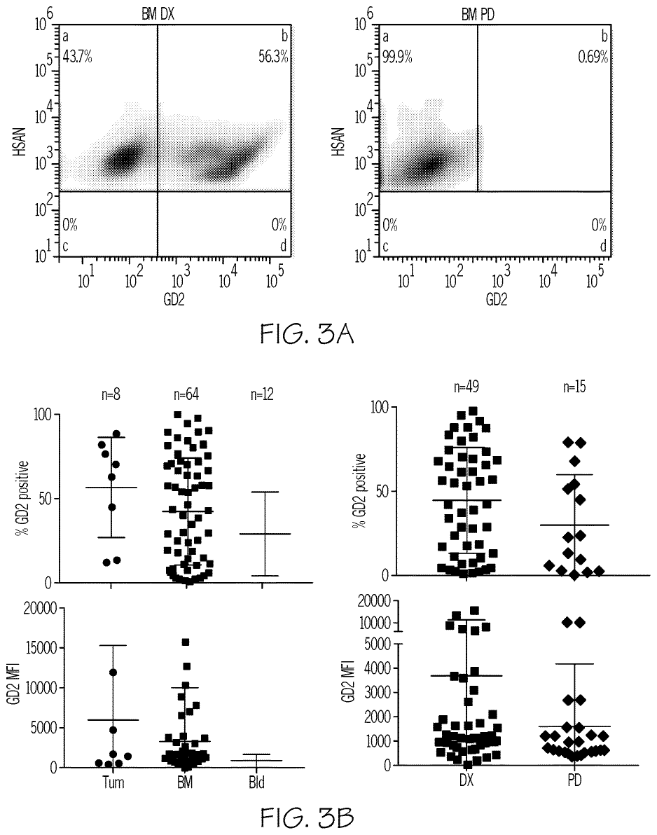

Preliminary data demonstrated that this approach permits the reliable quantification of dinutuximab binding and, importantly, demonstrated a significant variation in the % of GD2 positive cells and/or GD2 antigen density between cell lines and patients (FIGS. 1A-1B and 3B). Applicant also observed that multiple NB PDCLs from patients at progressive disease had low GD2 expression, consistent with the decrease in GD2 expression at time of progressive disease observed previously.

### Example 1.3. Multicolor Flow Cytometry Assay to Quantify GD2 on NB Cells in Blood and Bone Marrow Aspirates

It is desirable to confirm observations obtained in cell lines and PDXs directly in patient samples. Also, as Applicant's cell line panel results suggest that low GD2 expression is not rare in progressive disease, it is likely that there are progressive disease patients currently receiving antibody without likely potential for clinical benefit. Therefore, methodologies suitable for clinical sample use are desirable.

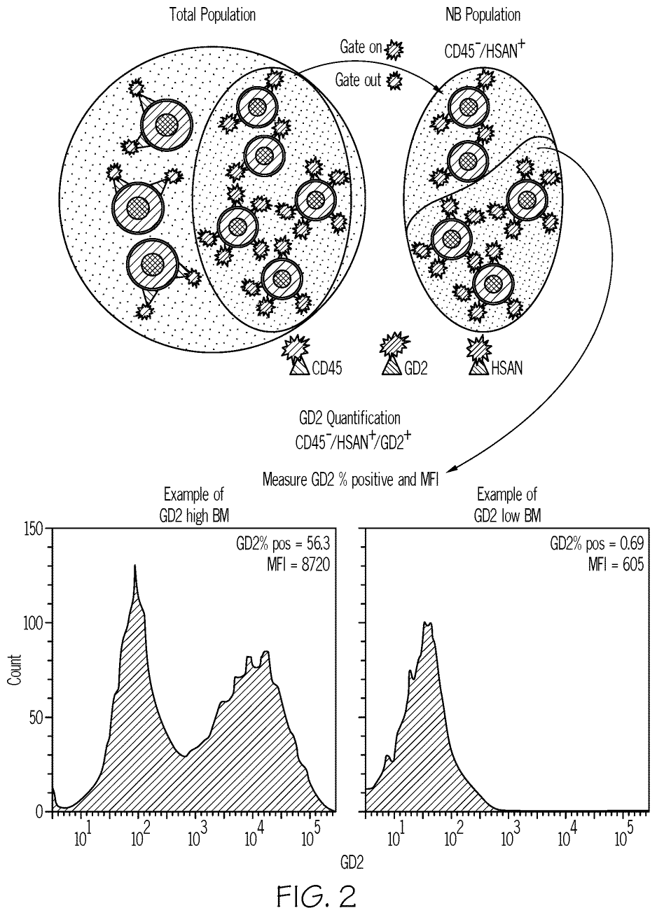

As the usual source of tumor cells at time of NB progression is marrow aspirates, Applicant developed a multicolor flow cytometry assay capable of quantifying dinutuximab-NB binding in marrow and blood (FIGS. 2-3D). Analyses are done on the day of sample receipt.

Previously, Applicant generated a monoclonal antibody, HSAN, that binds to a majority of NBs but does not bind to non-neuroblastoma cells in blood or bone marrow. Applicant has used the HSAN antibody as part of a cocktail of antibodies that enables sensitive detection of NB in bone marrow (due to the totally negative binding of HSAN or anti-GD2 to normal marrow elements). Applicant conjugated Alexa Fluor 488 (green) to HSAN to enable its use in a two color flow cytometry assay to gate on HSAN-positive NB cells and quantified dinutuximab binding by co-staining with Alexa Fluor 647 (red)-conjugated dinutuximab. The assay was validated using mixtures of NB cells into blood (data not shown but included in J. Clin. Med. 2023, 12, 6223).

FIG. 2 illustrates the multi-color assay which also employs Brilliant Violet 421 (blue violet)-labeled anti-CD45 ab to gate out hematopoietic cells. Applicant has used this assay on clinical samples provided by the COG. FIGS. 3A-3D demonstrate that this assay can quantitate dinutuximab binding to NB cells in tumor, blood, and bone marrow. Thus, Applicant's preliminary data demonstrate that Applicant's multi-color assay is capable of quantifying NB cell GD2 expression levels in patient marrow aspirates. Applicant observed a significantly lower intensity of binding in progressive disease patient samples compared to those obtained pre-therapy at diagnosis.

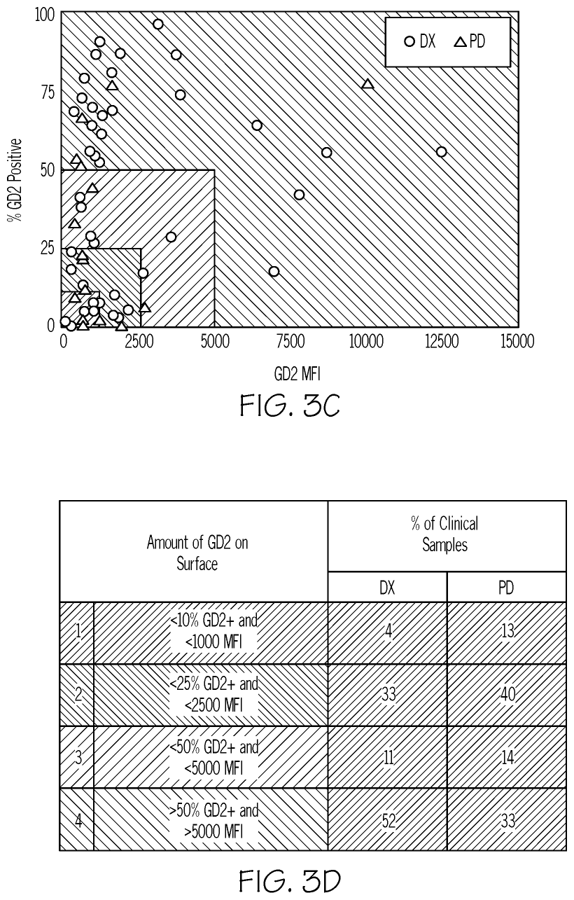

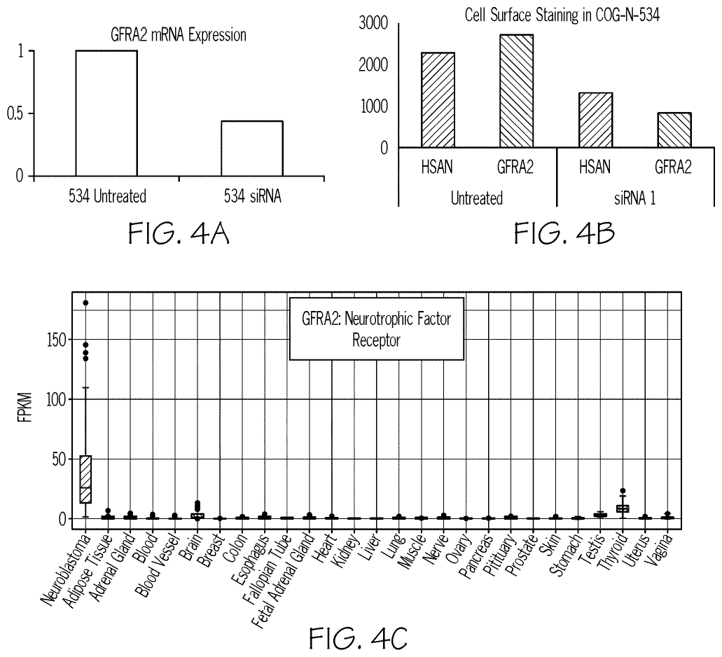

In no cases did Applicant identify GD2-positive cells that were negative for HSAN. The results using HSAN were confirmed by analyzing the same samples with another NB-specific antibody, 459, confirming that samples analyzed for GD2 were all NB cells. Applicant's data show that NB tumor cells from 37% to 53% of patients have very low or low binding to dinutuximab (FIGS. 4A-4C). These patients could potentially be receiving dinutuximab therapy without benefit.

FIGS. 1C-1D show binding patterns for HSAN compared to dinutuximab (GD2) across a panel of 24 neuroblastoma patient-derived cell lines (PDCLs) from the Children's Oncology Group (COG) repository established from 24 different patients. Most GD2-low and GD2 negative cell lines show strong HSAN binding.

### Example 1.4. Molecular Production of HSAN Antibodies

To enable production of the HSAN antibody using molecularly synthetic methods and also to enable humanizing HSAN to use for therapy of patients requires having the sequence of the combining site of the antibody that recognizes the antigen on the tumor cell surface. Applicant has recently sequenced the HSAN antibody protein, and also obtained the DNA sequence from the mRNA of the HSAN hybridoma that codes for the antibody. Both sequences produce the same predicted protein sequence. This will allow molecular production of HSAN and of a humanized HSAN antibody that can be used for diagnostic and therapeutic purposes.

### Example 1.5. Sequences Obtained from the HSAN Antibody

Table 1 provides light chain and heavy chain amino acid sequences of the HSAN antibody, including the complementarity determining regions (CDR).

TABLE 1
Amino acid Sequences of the HSAN antibody. Host species: Mus musculus.

| HSAN Antibody | |
|---|---|
| Fragment | Amino Acid Sequence |
| Light chain | DVVMTQTPLTLSVTIGQPASISCKSSQSLLDTDGKTYLNWLL |
| | QRPGQSPKRLIYLVSKLDSGVPDRFTGSGSGTDFTLKISRVE |
| | AEDLGVYYCWQGTHFPQTFGGGTKLEIKRADAAPTVSIFPP |
| | SSEQLTSGGASVVCFLNNFYPKDINVKWKIDGSERQNGVLN |
| | SWTDQDSKDSTYSMSSTLTLTKDEYERHNSYTCEATHKTST |
| | SPIVKSFNRNEC (SEQ ID NO: 1) |
| Heavy Chain | EVQLQQSGPELVKPGASVKISCKASGYSFTGYFMHWVKQR |
| | HVQSLEWIGRINPYNGATNYNQIFKDKASLTLDKSSSTAYM |
| | ELHSLTSEDSAVYYCASGGHYYGNVDYWGQGTTLTVSSAK |
| | TTPPSVYPLAPGSAAQTNSMVTLGCLVKGYFPEPVTVTWNS |
| | GSLSSGVHTFPAVLQSDLYTLSSSVTVPSSTWPSETVTCNVA |
| | HPASSTKVDKKIVPRDCGCKPCICTVPEVSSVFIFPPKPKDVL |
| | TITLTPKVTCVVVDISKDDPEVQFSWFVDDVEVHTAQTQPR |
| | EEQFNSTFRSVSELPIMHQDWLNGKEFKCRVNSAAFPAPIEK |
| | TISKTKGRPKAPQVYTIPPPKEQMAKDKVSLTCMITDFFPEDI |
| | TVEWQWNGQPAENYKNTQPIMDTDGSYFVYSKLNVQKSN |
| | WEAGNTFTCSVLHEGLHNHHTEKSLSHSPGK (SEQ ID NO: 2) |
| Heavy Chain Fragment | MGWSWIFLFLLSGTAGVLSEVQLQQSGPELVKPGASVKISC |
| | KASGYSFTGYFMHWVKQRHVQSLEWIGRINPYNGATNYNQ |
| | IFKDKASLTLDKSSSTAYMELHSLTSEDSAVYYCASGGHYY |
| | GNVDYWGQGTTLTVSS (SEQ ID NO: 3) |
| Light Chain Fragment | MSPAQFLLLLVLWIRETNGDVVMTQTPLTLSVTIGQPASISC |
| | KSSQSLLDTDGKTYLNWLLQRPGQSPKRLIYLVSKLDSGVP |
| | DRFTGSGSGTDFTLKISRVEAEDLGVYYCWQGTHFPQTFGG |
| | GTKLEIK (SEQ ID NO: 4) |
| CDR L1 Sequence | KSSQSLLDTDGKTYLN (SEQ ID NO: 5) |
| CDR L2 Sequence | LVSKLDS (SEQ ID NO: 6) |
| CDR L3 Sequence | WQGTHFPQT (SEQ ID NO: 7) |
| CDR H1 Sequence | GYFMH (SEQ ID NO: 8) |
| CDR H2 Sequence | RINPYNGATNYNQIFKD (SEQ ID NO: 9) |
| CDR H3 Sequence | GGHYYGNVDY (SEQ ID NO: 10) |
| CDR H1 Sequence | GYSFTGY (SEQ ID NO: 11) |
| CDR H2 Sequence | NPYNGA (SEQ ID NO: 12) |

Table 2 provides light chain and heavy chain DNA sequences of the HSAN antibody.

TABLE 2
DNA Sequences of the HSAN antibody. Host species: Mus musculus.

| HSAN Antibody | |
|---|---|
| Fragment | DNA Sequence |
| Light chain | ATGAGTCCTGCCCAGTTCCTGCTTCTGTTAGTGCTCTGGA |
| | TTCGGGAAACCAACGGTGATGTTGTGATGACCCAGACTC |
| | CACTCACTTTGTCGGTTACCATTGGACAACCAGCCTCCAT |
| | CTCTTGCAAGTCAAGTCAGAGCCTCTTAGACACTGATGG |
| | AAAGACATATTTGAATTGGTTATTACAGAGGCCAGGCCA |
| | GTCTCCAAAGCGCCTAATCTATCTGGTGTCTAAACTGGAC |
| | TCTGGAGTCCCTGACAGGTTCACTGGCAGTGGATCAGGG |
| | ACAGATTTCACACTGAAAATCAGCAGAGTGGAGGCTGAG |
| | GATTTGGGAGTTTATTATTGCTGGCAAGGTACACATTTTC |
| | CTCAGACGTTCGGTGGAGGCACCAAGCTGGAAATCAAA |
| | (SEQ ID NO: 13) |
| Heavy Chain | ATGGGATGGAGCTGGATCTTTCTCTTTCTCCTGTCAGGAA |
| | CTGCAGGTGTCCTCTCTGAGGTCCAGCTGCAACAGTCTGG |
| | ACCTGAGCTGGTGAAGCCTGGGGCTTCAGTGAAGATATC |
| | CTGCAAGGCTTCTGGTTACTCATTCACTGGCTACTTCATG |
| | CACTGGGTGAAGCAAAGGCATGTACAGAGCCTTGAGTGG |
| | ATTGGACGTATTAATCCTTACAATGGTGCTACTAACTACA |
| | ACCAGATTTTCAAAGACAAGGCCAGCTTGACTTTAGATA |
| | AGTCCTCCAGCACAGCCTACATGGAGCTCCACAGCCTGA |
| | CATCTGAGGACTCTGCAGTCTATTACTGTGCAAGCGGAG |
| | GGCATTACTACGGCAACGTTGACTACTGGGGCCAAGGCA |
| | CCACTCTCACAGTCTCCTCA (SEQ ID NO: 14) |

### Example 1.6. Defining the Antigen for the HSAN Antibody

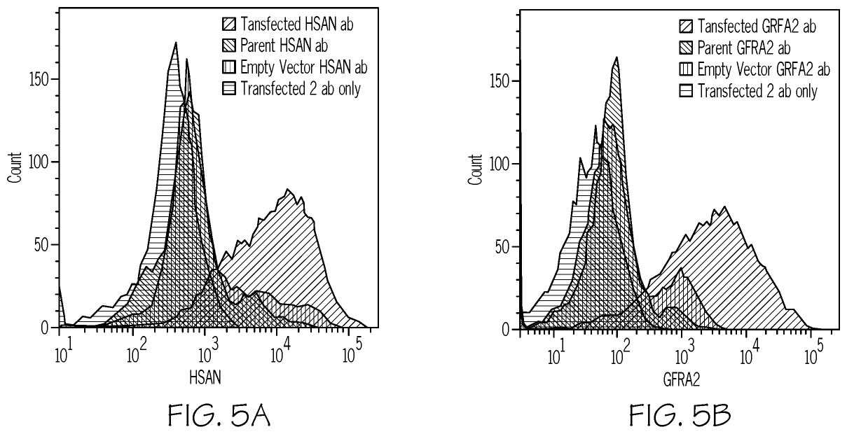

To employ HSAN for in vivo therapy of patients requires knowing the antigen. Defining the antigen for the HSAN antibody has been elusive, in part because it does not work with immunoblotting. It was known that HSAN bound to a protein antigen, but the number of possibilities for cell surface proteins on neuroblastoma are high in number. Applicant recently carried out mass spectrometry proteomics to identify the antigen bound by the HSAN antibody. Two independent mass spectrometry analyses were completed. One analysis was conducted from the fraction Applicant eluted off of beads following immunoprecipitation (IP) with HSAN. The second analysis was conducted from gel bands cut from running the HSAN IP product on a denaturing protein gel. Both data sets were analyzed independently and resulted in GDNF family receptor alpha 2 (GFRA2) being predicted as a target for HSAN. Multiple proteins were predicted, and GFRA2 scored as highly likely. The predicted molecular weight of GFRA2 is consistent with the ~50 kD band observed on a gel after HSAN IP (data not shown). However, to determine that HSAN binds to GFRA2 required confirmatory experiments (FIGS. 4A-4C and 5A-5B) that were motivated to be conducted by analyzing the mass spectrometry data together with known patterns of gene expression.

GFRA2 is a GPI-linked plasma membrane receptor for GDNF and neurturin that promotes neuroblastoma (NB) proliferation. RNA expression of GFRA2 is high in NB, with limited normal tissue expression (low in brain and thyroid, not expressed elsewhere). Applicant's RNA sequencing data show consistently high GFRA2 expression in NB patient-derived cell lines and PDX's (data not shown). Using RNA sequencing data, GFRA2 expression in NB patient tumors was found to be higher than 40 other cancer types, with Ewing's sarcoma having the second highest GFRA2. Applicant observed HSAN and GFRA2 surface staining in 2 of 3 Ewing's sarcoma cell lines (data not shown). In a paired antigen expression analysis, GFRA2 was predicted to be a suitable and safe target for CAR-T neuroblastoma therapy.

In NB cell lines surface staining of both GFRA2 (commercial polyclonal antibody) and HSAN correlated with mRNA expression of GFRA2 (data not shown). Applicant demonstrated that HSAN surface staining was decreased after siRNA GFRA2 knockdown (FIGS. 4A-4B).

Thus, Applicant's data indicate that GFRA2 is the antigen binding the HSAN antibody. HSAN is the first known anti-GFRA2 monoclonal antibody that was derived using neuroblastoma cells as the antigen. The expression pattern of GFRA2 indicate a strong potential for humanized HSAN as a therapeutic antibody for various cancers, such as neuroblastoma and Ewing's sarcoma.

As illustrated in FIGS. 5A-5B, Applicant conducted an additional experiment to demonstrate that HSAN binds to GFRA2. HEK293 cells (GFRA2-negative cells) were plated the day before transfection. Cells were then transfected with either GFRA2 plasmid DNA or empty vector control plasmid DNA using the MegaTran 2.0 plasmid transfection reagent. Medium was changed after 24 hours of transfection. GFRA2 surface expression was assessed by flow cytometry 72 hours post-transfection on a BD Fortessa flow cytometer. Untransfected parental HEK cells, empty vector transfected, or GFRA2 transfected cells were stained with the HSAN1.2 antibody directly conjugated to Alexa Fluor 488, a commercially available anti-GFRA2 antibody with an anti-goat CF 594 secondary antibody, or the secondary antibody alone. Flow data were analyzed using FlowJo software. The results in FIGS. 5A-5B demonstrate that cells that do not express GFRA2 were induced to express GFRA2 on the cell surface by transfection of GFRA2 DNA, detectable by an antibody to GFRA2 and also by the HSAN monoclonal antibody.

### Example 1.7. Conclusion

In sum, Applicant previously developed HSAN, a monoclonal antibody to human neuroblastomas. Recently, Applicant took advantage of the ability to sequence an antibody, thereby enabling molecular modification and production of HSAN for the first time. The sequence is being used to develop an antibody-drug conjugate.

Applicant also recently identified the antigen that HSAN binds to. Using mass spectrometry proteomics data from HSAN immunoprecipitation materials (from neuroblastoma cell lines and xenografts), Applicant identified a likely candidate. Subsequently, knock down of the gene for the candidate antigen (GFRA2) established GFRA2 as the antigen bound by HSAN. To Applicant's knowledge, the aforementioned antibody is the first monoclonal antibody generated with neuroblastoma cells as the antigen that binds to GFRA2, which has been speculated to be an ideal target for immunotherapy of neuroblastoma and Ewing's sarcoma.

The antibody drug conjugate with the HSAN antibody could be developed for treating children with neuroblastoma, as could a humanized version of the antibody itself. Preliminary data show that the antibody binds to tumor cells from patients resistant to the current FDA approved antibody for neuroblastoma, which is against GD2. The marketplace now supports 3 different FDA approved commercial antibodies to GD2, only used for neuroblastoma. HSAN can also be used as a diagnostic reagent for neuroblastoma and other GFRA2 positive tumors, such as Ewing's sarcoma.

Applicant has assessed binding of GD2 antibodies and HSAN to tumor cells in bone marrow from children with neuroblastoma. Applicant has now analyzed over 100 patients and HSAN is more consistently binding to the tumor cells than GD2 antibodies.

Loss of binding (more frequent in neuroblastoma after treatment with GD2 antibodies) as a mechanism of cancer cell resistance to immunotherapy. HSAN could be effective against neuroblastomas resistant to anti-GD2 antibodies. HSAN is also an important diagnostic reagent in that one can use to identify neuroblastoma cells in blood or bone marrow to enable quantifying the amount of GD2 on those neuroblastoma cells as a mechanism of resistance to dinutuximab.

### Example 2. Development of a Humanized GFRA2-Targeting Antibody Drug Conjugate for Neuroblastoma (NB), and Ewing Sarcoma (EWS)

In this Example, Applicants describe the development of a proteogenomic approach to identify high confidence cell surface proteins for immunotherapy development. Applicants applied this approach to neuroblastoma (NB) and Ewing sarcoma (EWS)-often fatal childhood cancers.

In particular, Applicants recently defined the NB/EWS surfaceomes using integrative proteogenomics to prioritize proteins as candidate immunotherapeutic targets. GFRA2 was a top ranked candidate for both NB and EWS.

This Example aimed to (1) validate and assess the mechanism of GFRA2 overexpression, (2) identify selective antibody binders to GFRA2, and (3) engineer antibody drug conjugates (ADCs) and test for internalization and potency.

### Example 2.1. Validation of Cell Surface Expression of GFRA2 in NB

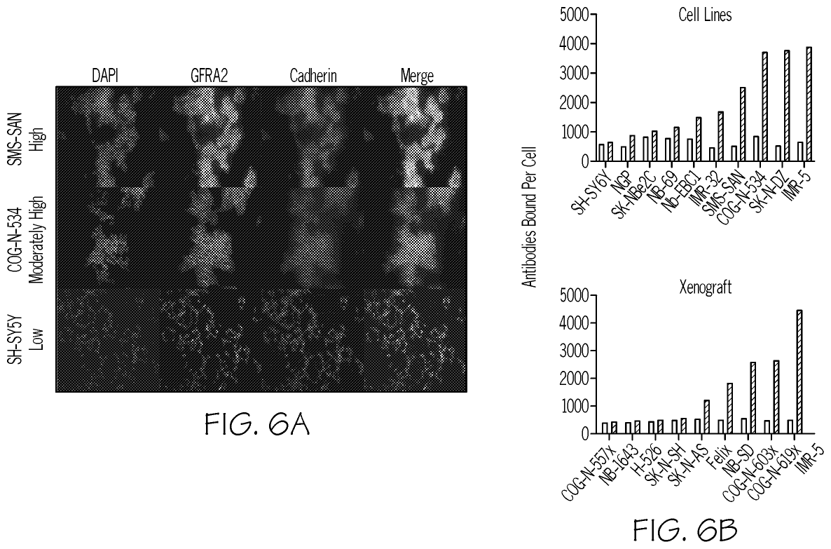

Applicants validated the cell surface expression of GFRA2 in NB cells. The results are shown in FIGS. 6A-6B. FIG. 6A shows the immunofluorescence of neuroblastoma cells, where it shows colocalization of GFRA2 with cell surface protein Cadherin. FIG. 6B shows flow cytometry results, where cell surface expression of GRFA2 in subsets of cell lines and xenografts are shown.

### Example 2.2. GFRA2 is Expressed in EWS and is a EWSR1::FLI1 Target Gene

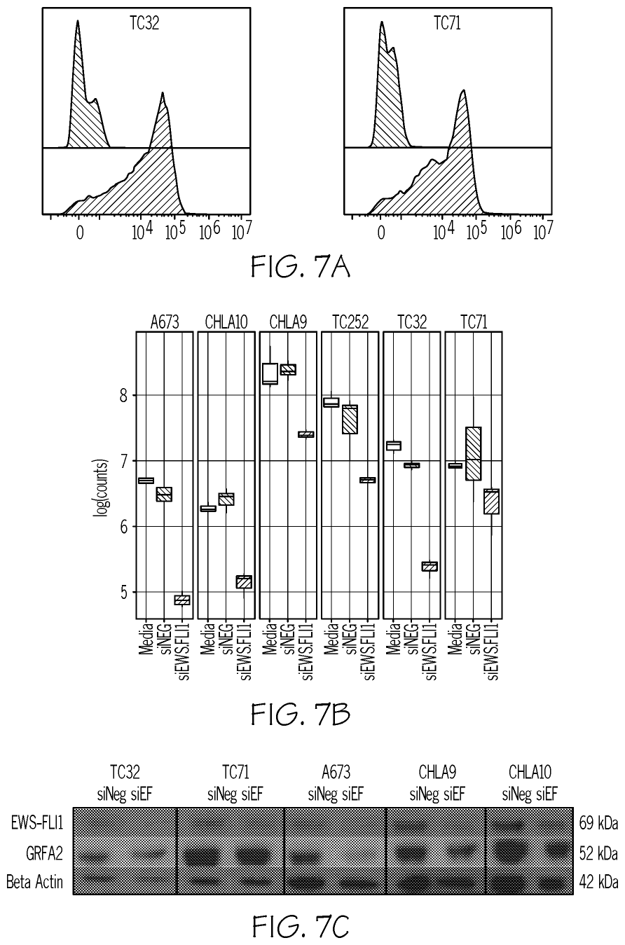

Applicants also established that GRFA2 is expressed in EWS cell lines and is a EWSR::FLI1 target gene. The results are shown in FIGS. 7A-7C. FIG. 7A shows cell surface validation of GFRA2 in EWS cell lines (N=4) by flow cytometry (CHLA9, CHLA10 not shown). FIG. 7B shows that siRNA knockdown of EWSR1::FLI1 results in down-regulation of GFRA2, suggesting that GFRA2 is a direct EWSR1::FLI1 transcriptional target. FIG. 7C provides western blot results validating GFRA2 knockdown in response to siEWSR1::FLI1 at the protein level.

### Example 2.3. Humanization of HSAN Antibody Targeting GFRA2

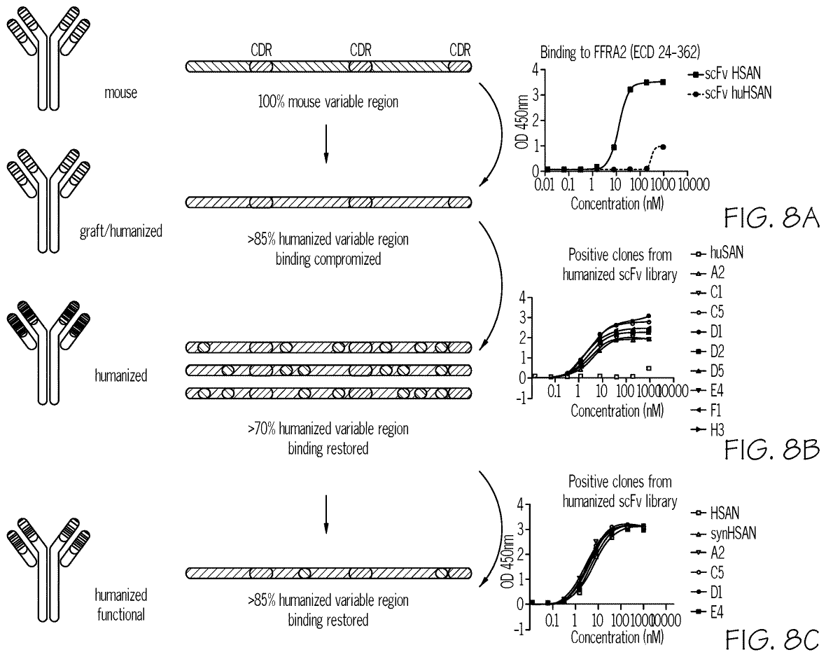

As illustrated in FIGS. 8A-8C, Applicants humanized an HSAN antibody targeting GFRA2. FIG. 8A shows that CDR graft humanization from mouse to humans compromised binding. FIG. 8B demonstrates that back mutations from mouse to human identified regions are critical for binding. FIG. 8C illustrates that a combination of sequences yielded the synthetic humanized HSAN (SynHuHSAN) that is ~94% human.

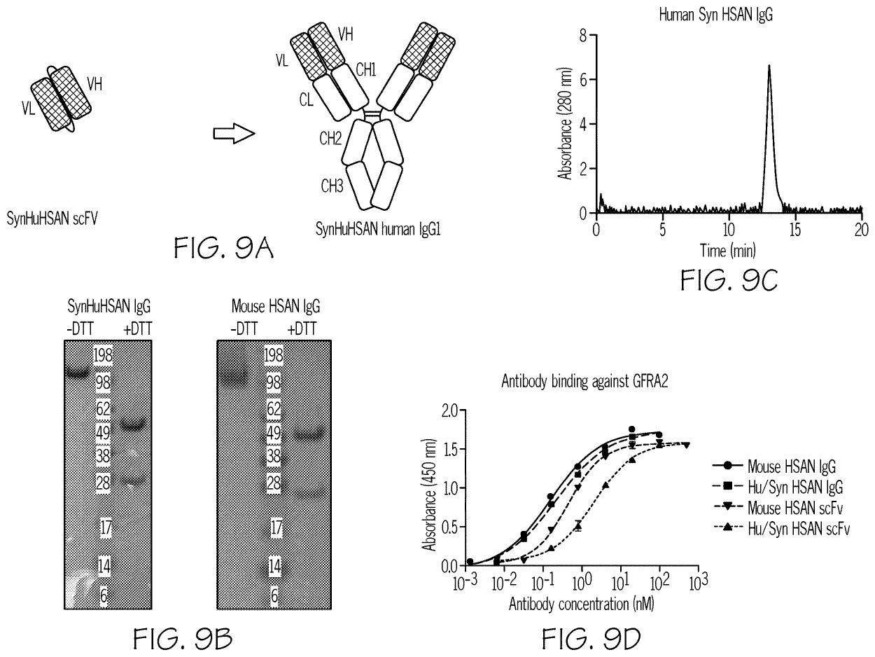

FIGS. 9A-9D illustrate the reformatting of SynHuHSAN scFv into human IgG1 and comparison of binding affinity of SynHuHSAN with the parental murine HSAN by ELISA. FIG. 9A illustrates the reformatting of scFv SynHuHSAN into IgG1. FIG. 9B shows an SDS-PAGE of SynHuHSAN and murine HSAN under both reducing and non-reducing conditions. FIG. 9C shows the size exclusion chromatography profile for IgG1 SynHuHSAN. The single peak indicates good monomeric folding for IgG1 SynHuHSAN. FIG. 9D shows the results of a binding assay against recombinant human GFRA2 ECD protein by HSAN antibody variants.

### Example 2.4. Humanized Antibody Sequences

Table 3 provides a summary of the sequences of the humanized GFRA2 antibodies.

TABLE 3
Amino acid sequences of the humanized GFRA2 antibodies.

| HSAN Antibody | |
|---|---|
| Portion | Amino Acid Sequence |
| CDR L1 Sequence | KSSQSLLDTDGKTYLN (SEQ ID NO: 15) |
| CDR L2 Sequence | LVSKLDS (SEQ ID NO: 16) |
| CDR L3 Sequence | WQGTHFPQT (SEQ ID NO: 17) |
| CDR H1 Sequence | GYFMH (SEQ ID NO: 18) |
| CDR H2 Sequence | RINPYNGATNYNQIFKD (SEQ ID NO: 19) |
| CDR H3 Sequence | GGHYYGNVDY (SEQ ID NO: 20) |
| CDR H1 Sequence | GYSFTGY (SEQ ID NO: 21) |
| CDR H2 Sequence | NPYNGA (SEQ ID NO: 22)) |
| Full-length Antibody | QVQLVQSGPEVKKPGASVKVSCKASGYTFTGYFMHWVKQ |
| (A2-HB2151) | TPGQGLEWIGRINPYNGATNYNQIFKDRVTMTLDKSSSTAY |
| | MELHSLTSDDTAVYYCASGGHYYGNVDYWGQGTLLTVSS |
| | GGGGSGGGGSGGGGSDVVMTQSPLSLSVTLGQPASISCKSS |
| | QSLLDTDGKTYLNWLQQRPGQSPRRLIYLVSKLDSGVPDRF |
| | SGSGSGTDFTLKISRVEAEDVGVYYCWQGTHFPQTFGGGTK |
| | LEIK (SEQ ID NO: 23) |
| Full-length Antibody | QVQLVQSGPEVKKPGASVKISCKASGYTFTGYFMHWVKQT |
| (C1-HB2151) | HGQGLEWIGRINPYNGATNYNQIFKDRVTLTLDTSSSTAYM |
| | ELPRLRSDDTAVYYCASGGHYYGNVDYWGQGTLVTVSSG |
| | GGGSGGGGSGGGGSDVVMTQSPLSLPVTLGQPASISCKSSQ |
| | SLLDTDGKTYLNWLLQRPGQSPRRLIYLVSKLDSGVPDRFS |
| | GSGSGTDFTLKISRVEAEDVGVYYCWQGTHFPQTFGGGTKL |
| | EIK (SEQ ID NO: 24) |
| Full-length Antibody | QVQLVQSGAEVIKPGASVKISCKASGYTFTGYFMHWVKQT |
| (C5-HB2151) | PGQGLEWMGRINPYNGATNYNQIFKDRVTLTLDTSSSTAY |
| | MELSRLRSDDTAVYYCASGGHYYGNVDYWGQGTLLTVSS |
| | GGGGSGGGGSGGGGSDVVMTQSPLSLPVTLGQPASISCKSS |
| | QSLLDTDGKTYLNWLQQRPGQSPRRLIYLVSKLDSGVPDRF |
| | SGSGSGTDFTLKISRVEAEDVGVYYCWQGTHFPQTFGGGTK |
| | LEIK (SEQ ID NO: 25) |
| Full-length Antibody | QVQLVQSGPEVIKPGASVKVSCKASGYTFTGYFMHWVRQT |
| (D1-HB2151) | PGQGLEWIGRINPYNGATNYNQIFKDRVTMTLDTSSSTAYM |
| | ELSSLRSDDTAVYYCASGGHYYGNVDYWGQGTLVTVSSG |
| | GGGSGGGGSGGGGSDVVMTQSPLSLSVTLGQPASISCKSSQ |
| | SLLDTDGKTYLNWLQQRPGQSPRRLIYLVSKLDSGVPDRFS |
| | GSGSGTDFTLKISRVEAEDVGVYYCWQGTHFPQTFGGGTKL |
| | EIK (SEQ ID NO: 26) |
| Full-length Antibody | QVQLVQSGPEVKKPGASVKVSCKASGYTFTGYFMHWVKQ |
| (D2-HB2151) | TPGQGLEWIGRINPYNGATNYNQIFKDRVTLTLDTSISTAYM |
| | ELPRLRSDDTAVYYCASGGHYYGNVDYWGQGTLLTVSSG |
| | GGGSGGGGSGGGGSDVVMTQSPLSLSVTLGQPASISCKSSQ |
| | SLLDTDGKTYLNWLLQRPGQSPRRLIYLVSKLDSGVPDRFS |
| | GSGSGTDFTLKISRVEAEDVGVYYCWQGTHFPQTFGGGTKL |
| | EIK (SEQ ID NO: 27) |
| Full-length Antibody | QVQLVQSGPEVKKPGASVKVSCKASGYSFTGYFMHWVRQ |
| (D5-HB2151) | THGQGLEWIGRINPYNGATNYNQIFKDRVTLTLDKSISTAY |
| | MELSRLTSDDTAVYYCASGGHYYGNVDYWGQGTLVTVSS |
| | GGGGSGGGGSGGGGSDVVMTQSPLSLSVTLGQPASISCKSS |
| | QSLLDTDGKTYLNWLQQRPGQSPRRLIYLVSKLDSGVPDRF |
| | SGSGSGTDFTLKISRVEAEDVGVYYCWQGTHFPQTFGGGTK |
| | LEIK (SEQ ID NO: 28) |
| Full-length Antibody | QVQLVQSGPEVIKPGASVKISCKASGYTFTGYFMHWVRQAP |
| (E4-HB2151) | GQGLEWIGRINPYNGATNYNQIFKDRVTLTLDTSSSTAYME |
| | LSRLRSDDTAVYYCASGGHYYGNVDYWGQGTLLTVSSGG |
| | GGSGGGGSGGGGSDVVMTQSPLSLSVTLGQPASISCKSSQS |
| | LLDTDGKTYLNWLLQRPGQSPRRLIYLVSKLDSGVPDRFSG |
| | SGSGTDFTLKISRVEAEDVGVYYCWQGTHFPQTFGGGTKLE |
| | IK (SEQ ID NO: 29) |
| Full-length Antibody | QVQLVQSGPEVIKPGASVKISCKASGYTFTGYFMHWVKQT |
| (F1-HB2151) | HGQGLEWMGRINPYNGATNYNQIFKDRVTMTLDKSSSTAY |
| | MELSSLRSDDTAVYYCASGGHYYGNVDYWGQGTLVTVSS |
| | GGGGSGGGGSGGGGSDVVMTQSPLSLPVTLGQPASISCKSS |
| | QSLLDTDGKTYLNWLLQRPGQSPRRLIYLVSKLDSGVPDRF |
| | SGSGSGTDFTLKISRVEAEDVGVYYCWQGTHFPQTFGGGTK |
| | LEIK (SEQ ID NO: 30) |
| Full-length Antibody | QVQLVQSGPEVIKPGASVKVSCKASGYTFTGYFMHWVRQT |
| (H3-HB2151) | PGQGLEWIGRINPYNGATNYNQIFKDRVTLTLDKSSSTAYM |
| | ELSSLTSDDTAVYYCASGGHYYGNVDYWGQGTLVTVSSGG |
| | GGSGGGGSGGGGSDVVMTQSPLSLSVTLGQPASISCKSSQS |
| | LLDTDGKTYLNWLLQRPGQSPRRLIYLVSKLDSGVPDRFSG |
| | SGSGTDFTLKISRVEAEDVGVYYCWQGTHFPQTFGGGTKLE |
| | IK (SEQ ID NO: 31) |
| Full-length Antibody | QVQLVQSGPEVKKPGASVKVSCKASGYTFTGYFMHWVKQ |
| (Synthetic humanized | TPGQGLEWIGRINPYNGATNYNQIFKDRVTMTLDTSSSTAY |
| HSAN (synhuHSAN)) | MELSRLRSDDTAVYYCASGGHYYGNVDYWGQGTLVTVSS |
| | GGGGSGGGGSGGGGSDVVMTQSPLSLPVTLGQPASISCKSS |
| | QSLLDTDGKTYLNWLQQRPGQSPRRLIYLVSKLDSGVPDRF |
| | SGSGSGTDFTLKISRVEAEDVGVYYCWQGTHFPQTFGGGTK |
| | LEIK (SEQ ID NO: 32) |

### Example 2.5. Internalization of GFRA2 Directed HSAN Antibody

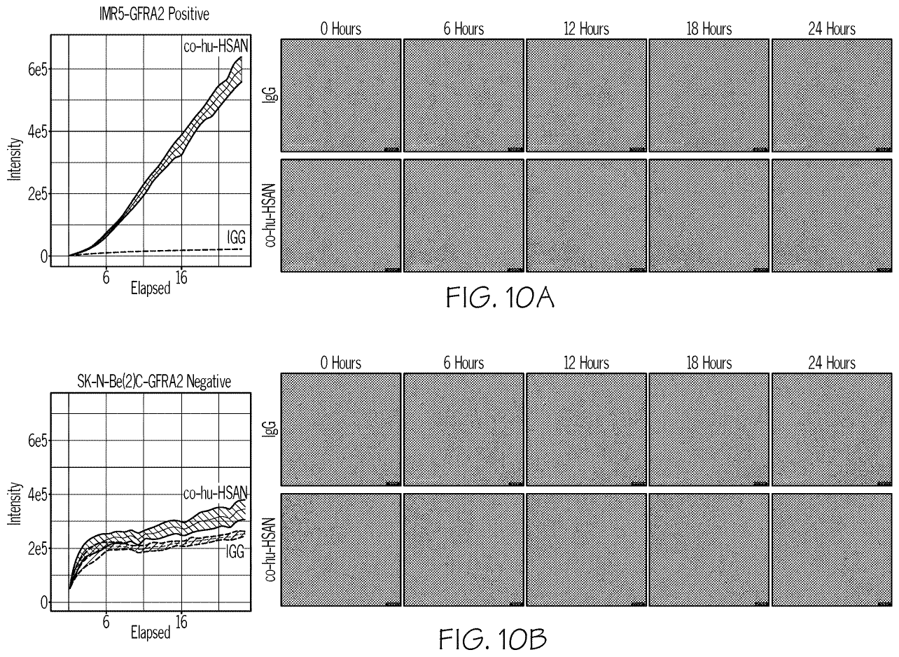

Applicants tested the internalization of the humanized HSAN antibody. The results are shown in FIGS. 10A-10B. SynhuHSAN IgG1 was conjugated to pH sensitive Fab-flour from Saurtorius. The results in FIG. 10A show that IMR5 (GFRA2+ NB) internalizes SynhuHSAN IgG1, as shown by fluorescence over time that is absent in the IgG control. The results in FIG. 10B show that SK-N-Be (2) C (GFRA2-NB) does not internalize the HSAN antibody as reflected in no fluorescence in either HSAN or IgG control.

### Example 2.6. Cell Surface Binding of SynHuHSAN IgG

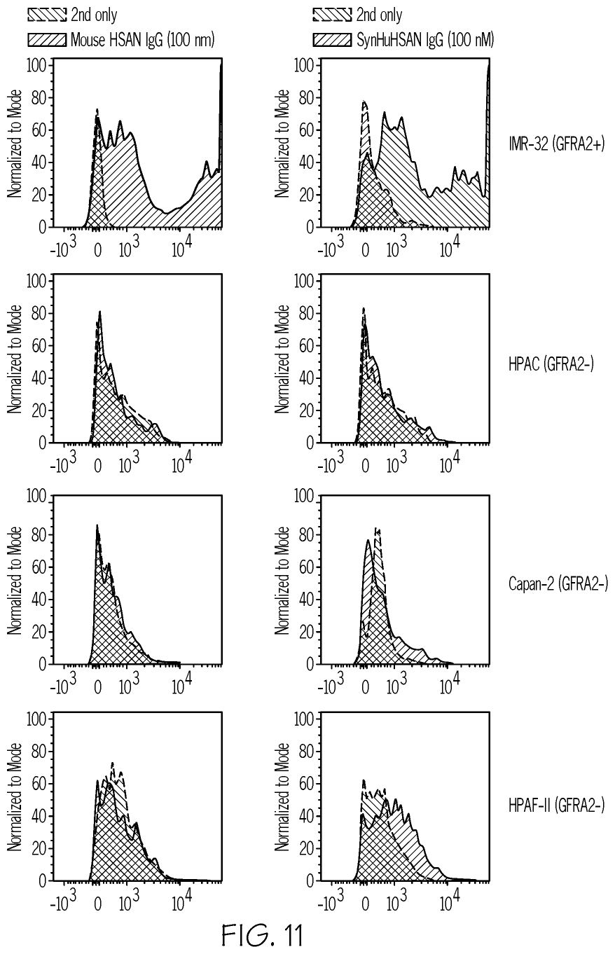

Additionally, Applicants tested the cell binding specificity for SynhuHSAN IgG1 by flow cytometry. The results are shown in FIG. 11. The results demonstrate that SynhuHSAN exhibits specific binding to GFRA2 positive cell line IMR-32, and non-existent or marginal binding to GFRA2 negative cell lines.

### Example 2.7. GFRA2-Directed ADC Shows Potent and Specific Cytotoxicity

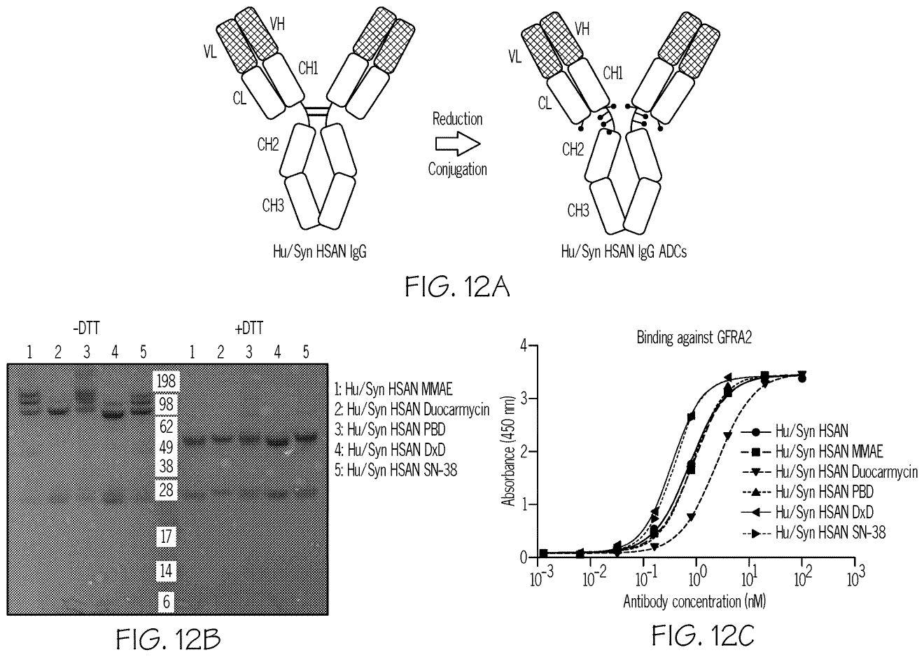

Applicants also demonstrated that GRFA2-directed antibody drug conjugates (ADCs) show potent and specific cytotoxicity. FIGS. 12A-12C show the generation of SynHuHSAN IgG ADCs through a reductive inter-disulfide bond derived thiol-maleimide conjugation method. FIG. 12A illustrates a scheme for the fabrication of the ADCs with the following drugs: monomethyl auristatin E (MMAE), Duocarmycin, pyrrolobenzodiazepine (PBD), deruxtecan (Dxd), and 7-ethyl-10-hydroxycamptothecin (SN-38). The maleimide functionality of each was conjugated into SynHuHSAN IgG. FIG. 12B shows an SDS-PAGE for the ADCs, indicating the intactness of HC and LC post conjugation. FIG. 12C shows the antigen binding capacities of the ADCs post conjugation, as tested by ELISA.

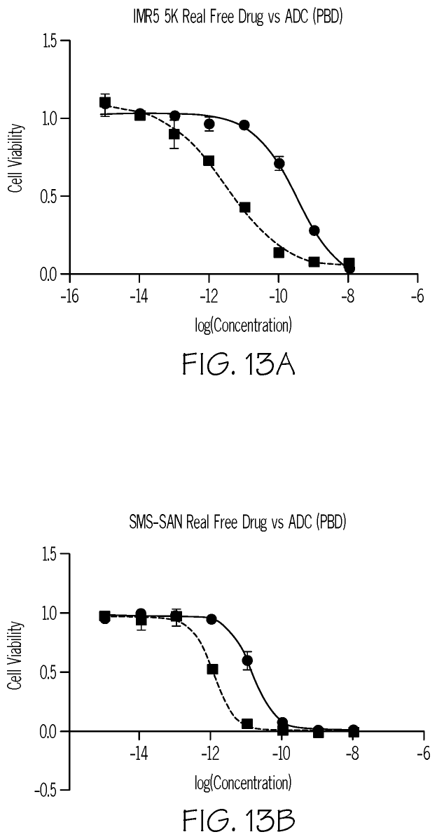

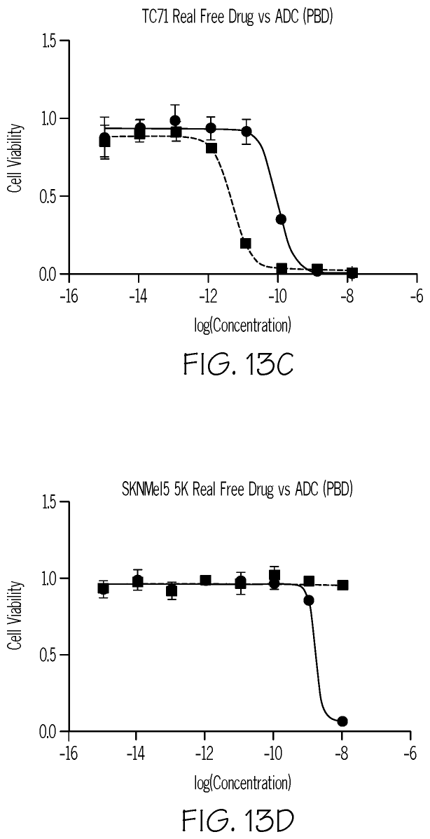

Additional results are summarized in FIGS. 13A-13D. In particular, SynhuHSAN IgG1 was conjugated to PBD to form a GFRA2 directed antibody drug conjugate (GFRA2-ADC). As demonstrated in the cell viability assays in FIGS. 13A-13B, GFRA2-ADC shows potent cytotoxicity in GFRA2+ neuroblastoma cell lines compared to PBD control. Additionally, as demonstrated in the cell viability assay in FIGS. 13C-13D, GFRA2-ADC shows potent cytotoxicity in GFRA2+ EWS cell line compared to PBD control (FIG. 13C), but no cytotoxicity in GFRA2-melanoma cell lines with PBD control showing potent cytotoxicity (FIG. 13D).

### Example 2.8. Discussion

In this Example, Applicants identified GFRA2 as an immunotherapeutic target, validated GFRA2 expression on the cell surface of NB and EWS, performed humanization of HSAN, verified internalization of HSAN, and developed an ADC with various drug payloads that induce cytotoxicity. The developed antibodies represent potential therapeutics for treating or preventing NB and EWS.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A humanized antibody, wherein the antibody binds to GDNF family receptor alpha 2 (GFRA2) and comprises one or more of the following complementarity determining regions (CDRs):
KSSQSLLDTDGKTYLN (SEQ ID NO: 15) or a sequence with at least 95% sequence identity to SEQ ID NO: 15,
LVSKLDS (SEQ ID NO: 16) or a sequence with at least 95% sequence identity to SEQ ID NO: 16,
WQGTHFPQT (SEQ ID NO: 17) or a sequence with at least 95% sequence identity to SEQ ID NO: 17,
GYFMH (SEQ ID NO: 18) or a sequence with at least 95% sequence identity to SEQ ID NO: 18,
RINPYNGATNYNQIFKD (SEQ ID NO: 19) or a sequence with at least 95% sequence identity to SEQ ID NO: 19,
GGHYYGNVDY (SEQ ID NO: 20) or a sequence with at least 95% sequence identity to SEQ ID NO: 20,
GYSFTGY (SEQ ID NO: 21) or a sequence with at least 95% sequence identity to SEQ ID NO: 21, and
NPYNGA (SEQ ID NO: 22) or a sequence with at least 95% sequence identity to SEQ ID NO: 22.

2. The antibody of claim 1, wherein the antibody is in a modified form selected from the group consisting of a chimeric form, a drug conjugated form, a bispecific form, an immunocytokine form, a labeled form, a fragmented form, or combinations thereof.

3. The antibody of claim 1, wherein the antibody comprises the following CDRs:
SEQ ID NO: 15,
SEQ ID NO: 16,
SEQ ID NO: 17,
SEQ ID NO: 18,
SEQ ID NO: 19, and
SEQ ID NO: 20.

4. The antibody of claim 1, wherein the antibody comprises the following CDRs:
SEQ ID NO: 15,
SEQ ID NO: 16,
SEQ ID NO: 17,
SEQ ID NO: 19,
SEQ ID NO: 20, and
SEQ ID NO: 21.

5. The antibody of claim 1, wherein the antibody is associated with a drug to form an antibody-drug conjugate.

6. The antibody of claim 5, wherein the drug comprises an anti-cancer drug.

7. The antibody of claim 6, wherein the anti-cancer drug is selected from the group consisting of pyrrolobenzodiazepine (PBD), monomethyl auristatin E (MMAE), deruxtecan (DXD), 7-ethyl-10-hydroxycamptothecin (SN38), duocarmycins, or combinations thereof.

8. The antibody of claim 1, wherein the antibody comprises one of the following sequences:
SEQ ID NO: 23 or a sequence with at least 95% sequence identity to SEQ ID NO: 23,
SEQ ID NO: 24 or a sequence with at least 95% sequence identity to SEQ ID NO: 24,
SEQ ID NO: 25 or a sequence with at least 95% sequence identity to SEQ ID NO: 25,
SEQ ID NO: 26 or a sequence with at least 95% sequence identity to SEQ ID NO: 26,
SEQ ID NO: 27 or a sequence with at least 95% sequence identity to SEQ ID NO: 27,
SEQ ID NO: 28 or a sequence with at least 95% sequence identity to SEQ ID NO: 28,
SEQ ID NO: 29 or a sequence with at least 95% sequence identity to SEQ ID NO: 29,
SEQ ID NO: 30 or a sequence with at least 95% sequence identity to SEQ ID NO: 30,
SEQ ID NO: 31 or a sequence with at least 95% sequence identity to SEQ ID NO: 31, or
SEQ ID NO: 32 or a sequence with at least 95% sequence identity to SEQ ID NO: 32.

9. A method of treating or preventing a cancer in a subject, said method comprising:
administering to the subject a humanized antibody, wherein the antibody binds to GDNF family receptor alpha 2 (GFRA2).

10. The method of claim 9, wherein the cancer is selected from the group consisting of GFRA2 positive cancers, neuroblastomas, Ewing's sarcoma, thyroid cancer, pediatric cancers, adult cancers, or combinations thereof.

11. The method of claim 9, wherein the antibody comprises one or more of the following complementarity determining regions (CDRs):
KSSQSLLDTDGKTYLN (SEQ ID NO: 15) or a sequence with at least 95% sequence identity to SEQ ID NO: 15,
LVSKLDS (SEQ ID NO: 16) or a sequence with at least 95% sequence identity to SEQ ID NO: 16,
WQGTHFPQT (SEQ ID NO: 17) or a sequence with at least 95% sequence identity to SEQ ID NO: 17,
GYFMH (SEQ ID NO: 18) or a sequence with at least 95% sequence identity to SEQ ID NO: 18,
RINPYNGATNYNQIFKD (SEQ ID NO: 19) or a sequence with at least 95% sequence identity to SEQ ID NO: 19,
GGHYYGNVDY (SEQ ID NO: 20) or a sequence with at least 95% sequence identity to SEQ ID NO: 20,
GYSFTGY (SEQ ID NO: 21) or a sequence with at least 95% sequence identity to SEQ ID NO: 21, and
NPYNGA (SEQ ID NO: 22) or a sequence with at least 95% sequence identity to SEQ ID NO: 22.

12. The method of claim 9, wherein the antibody comprises the following CDRs:
SEQ ID NO: 15,
SEQ ID NO: 16,
SEQ ID NO: 17,
SEQ ID NO: 18,
SEQ ID NO: 19, and
SEQ ID NO: 20.

13. The method of claim 9, wherein the antibody comprises the following CDRs:
SEQ ID NO: 15,
SEQ ID NO: 16,
SEQ ID NO: 17,
SEQ ID NO: 19,
SEQ ID NO: 20, and
SEQ ID NO: 21.

14. The method of claim 9, wherein the antibody is associated with a drug to form an antibody-drug conjugate.

15. The method of claim 14, wherein the drug comprises an anti-cancer drug.

16. The method of claim 15, wherein the anti-cancer drug is selected from the group consisting of pyrrolobenzodiazepine (PBD), monomethyl auristatin E (MMAE), deruxtecan (DXD), 7-ethyl-10-hydroxycamptothecin (SN38), duocarmycins, or combinations thereof.

17. The method of claim 9, wherein the antibody comprises one of the following sequences:
SEQ ID NO: 23 or a sequence with at least 95% sequence identity to SEQ ID NO: 23,
SEQ ID NO: 24 or a sequence with at least 95% sequence identity to SEQ ID NO: 24,
SEQ ID NO: 25 or a sequence with at least 95% sequence identity to SEQ ID NO: 25,
SEQ ID NO: 26 or a sequence with at least 95% sequence identity to SEQ ID NO: 26,
SEQ ID NO: 27 or a sequence with at least 95% sequence identity to SEQ ID NO: 27,
SEQ ID NO: 28 or a sequence with at least 95% sequence identity to SEQ ID NO: 28,
SEQ ID NO: 29 or a sequence with at least 95% sequence identity to SEQ ID NO: 29,
SEQ ID NO: 30 or a sequence with at least 95% sequence identity to SEQ ID NO: 30,
SEQ ID NO: 31 or a sequence with at least 95% sequence identity to SEQ ID NO: 31, or
SEQ ID NO: 32 or a sequence with at least 95% sequence identity to SEQ ID NO: 32.
